(19) United States
(12) Patent Application Publication
(10) Pub. No.: US 2021/0206744 A1
(43) Pub. Date: Jul. 8, 2021

(54) BENZIMIDAZOLE COMPOUNDS AS AGRICULTURAL CHEMICALS

(71) Applicant: REDAG CROP PROTECTION LTD, Wigan (GB)

(72) Inventors: Christopher John URCH, Wigan (GB); Roger John BUTLIN, Wigan (GB); Stephania CHRISTOU, Wigan (GB); Rebecca Kathryn BOOTH, Wigan (GB)

(21) Appl. No.: 16/755,396

(22) PCT Filed: Oct. 17, 2018
(86) PCT No.: PCT/GB2018/052988

§ 371 Date: Apr. 10, 2020

(30) Foreign Application Priority Data
Oct. 18, 2017 (GB) 1717143.0
May 23, 2018 (GB) 1808447.5

(51) Int. Cl. C07D 401/14; C07D 417/14; A01N 43/78; A01N 43/713; A01N 47/20

(52) U.S. Cl. CPC C07D 401/14; C07D 417/14; A01N 47/20; A01N 43/713; A01N 43/78

(57) ABSTRACT

The present invention relates to substituted benzimidazoles and related compounds which are of use in the field of agriculture as fungicides.

The present invention relates to substituted benzimidazoles and related compounds which are of use in the field of agriculture as fungicides.

Given the global increase in demand for food, there is an international need for new treatments to reduce food crop losses to disease, insects and weeds. Over 40% of crops are lost before harvest, and 10% post harvest, worldwide. Losses have actually increased since the mid-1990s.

A new threat contributing to this is the emergence of chemical-resistant organisms, for example, glyphosate-resistant weeds in USA and strobilurin-resistant strains of Septoria fungal species.

Recent research also suggests that the geographical spread of many crop pests and diseases is increasing, possibly as a result of global warming.

WO2012/136581 and WO2016/055802 provide a range of compounds that have proved active as fungicides.

An aim of certain embodiments of the present invention is to provide pesticides (e.g. fungicides) which have activity either non-selectively, i.e. broad spectrum activity, or which are active specifically against selective target organisms.

An aim of certain embodiments of the present invention is to provide compounds which are less persistent in the environment after use than prior art compounds. Alternatively or additionally the compounds of the present invention may be less prone to bioaccumulation once in the food chain than prior art compounds.

Another aim of certain embodiments of the invention is to provide compounds which are less harmful to humans than prior art compounds.

Alternatively or additionally, the compounds of the invention may be less harmful than prior art compounds to one or more of the following groups: amphibians, fish, mammals (including domesticated animals such as dogs, cats, cows, sheep, pigs, goats, etc.), reptiles, birds, and beneficial invertebrates (e.g. bees and other insects, or worms), beneficial nematodes, beneficial fungi and nitrogen-fixing bacteria.

Certain compounds of the invention may be as active or more active than prior art compounds. They may have activity against organisms which have developed a resistance to prior art compounds. However, certain embodiments of the present invention may also concern compounds which have a lower level of activity relative to prior art compounds. These lower activity compounds are still effective as fungicides but may have other advantages relative to existing compounds such as, for example, a reduced environmental impact.

Certain compounds of the invention may be more selective than the parent, i.e. they may have better, similar or even slightly lower activity than the parent against target species but have a significantly lower activity against non-target species (e.g. the crops which are being protected).

Certain embodiments of the invention provide compounds that achieve one or more of the above aims. The compounds may be active in their own right or may metabolise or react in aqueous media to yield an active compound.

### SUMMARY OF THE INVENTION

In a first aspect of the invention is provided a compound of formula (I):

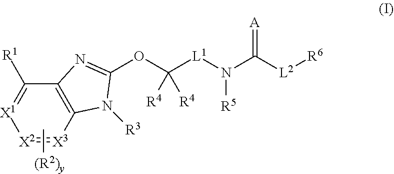

wherein X¹, X² and X³ are each selected from carbon and nitrogen; wherein no more than two of X¹, X² and X³ are nitrogen;

-L¹- is a heteroaryl group independently selected from 6-membered heteroaryl and 5-membered heteroaryl comprising at least one nitrogen atom in the ring, wherein said heteroaryl group is optionally substituted with from 1 to 3 R⁷ groups;

R¹ is independently selected from chloro, bromo, nitro, cyano, NR⁸R⁹, NR⁸CONR⁸R⁸, OR¹⁰, SR⁸, S(O)R⁸, OS(O)₂R⁸, S(O)₂R⁸, S(O)₂NR⁸R⁸, CO₂R⁸, C(O)R⁸, CONR⁸R⁸, C₁-C₆-alkyl, C₂-C₆-alkenyl, C₂-C₆-alkynyl, C₁-C₆-haloalkyl, phenyl, C₃-C₆-cycloalkyl, 3- to 6-membered heterocycloalkyl, 5- or 6-membered heteroaryl, and —O—C₁-C₄-haloalkyl;

R², R⁷ and R¹¹ are each independently at each occurrence selected from: halo, nitro, cyano, NR⁸R⁹, NR⁸CONR⁸R⁸, OR¹⁰, SR⁸, S(O)R⁸, OS(O)₂R⁸, S(O)₂R⁸, S(O)₂NR⁸R⁸, CO₂R⁸, C(O)R⁸, CONR⁸R⁸, C₁-C₆-alkyl, C₂-C₆-alkenyl, C₂-C₆-alkynyl, C₁-C₆-haloalkyl, phenyl, C₃-C₆-cycloalkyl, 3- to 6-membered heterocycloalkyl, 5- or 6-membered heteroaryl, and —O—C₁-C₄-haloalkyl;

R³ is independently selected from 5- or 6-membered heteroaryl, optionally further substituted with from 1 to 4 R¹¹ groups;

R⁴ and R¹³ are each independently at each occurrence selected from H, F, C₁-C₄-alkyl and C₁-C₄-haloalkyl;

or two R⁴ groups together with the carbon to which they are attached form a C₃-C₅-cycloalkyl group;

or two R¹³ groups together with the carbon to which they are attached form a C₃-C₅-cycloalkyl group;

R⁵, R⁸, R¹⁵ and R¹⁷ are each independently at each occurrence selected from H, C₁-C₆-alkyl and C₃-C₆-cycloalkyl;

or two R⁸ groups that are attached to the same nitrogen atom may form a 4- to 7-membered heterocycloalkyl ring;

R⁶ is independently selected from C₁-C₈-alkyl, C₁-C₈-haloalkyl, C₂-C₈-alkenyl, C₂-C₈-alkynyl, C₀-C₃-alkylene-R¹²; and —CR¹³R¹³L³R¹⁴;

R⁹ is independently at each occurrence selected from H, C₃-C₆-cycloalkyl, C₁-C₆-alkyl, C(O)—C₁-C₆-alkyl, C(O)O—C₁-C₆-alkyl and S(O)₂—C₁-C₆-alkyl;

or R⁸ and R⁹ together with the nitrogen to which they are attached form a 4- to 7-membered heterocycloalkyl ring;

R¹⁰ is independently at each occurrence selected from H, C₁-C₆-alkyl, C₃-C₆-alkenyl, C₃-C₆-alkynyl, C₁-C₆-haloalkyl, and C₀-C₃-alkylene-R^{10a}, wherein R^{10a} is independently selected from C₃-C₆-cycloalkyl and 3- to 6-membered heterocycloalkyl;

=A is independently selected from ═O and ═S;

-L²- is absent or is independently selected from —O—, —S—, and —NR¹⁵—;

R¹² and R¹⁶ are each independently at each occurrence selected from C₃-C₆-cycloalkyl, 3- to 6-membered heterocycloalkyl, 5- or 6-membered heteroaryl and phenyl;

R¹³ is independently at each occurrence selected from F, H and C₁-C₄-alkyl;

R¹⁴ is independently selected from H, C₁-C₃-alkyl, C₁-C₃-haloalkyl, C₃-C₃-alkenyl, C₃-C₃-alkynyl and C₀-C₃-alkylene-R¹⁶;

-L³- is independently selected from —O—, —S— and —NR¹⁷—;

y is an integer selected from 0, 1, 2 and 3;

wherein where any R¹-R¹⁷ group is or forms part of an alkyl, alkenyl, alkynyl, haloalkyl, cycloalkyl, heterocycloalkyl, phenyl or heteroaryl group, that alkyl, haloalkyl, cycloalkyl, heterocycloalkyl, phenyl or heteroaryl group is optionally substituted, where chemically possible, by 1 to 5 substituents which are each independently selected at each occurrence from: oxo, ═NR^{a}, ═NOR^{a}, halo, nitro, cyano, NR^{a}R^{b}, NR^{a}S(O)₂R^{a}, NR^{a}C(O)R^{a}, NR^{a}CONR^{a}R^{a}, NR^{a}CO₂R^{a}, OR^{a}, SR^{a}, S(O)R^{a}, S(O)₂R^{a}, S(O)₂NR^{a}R^{a}, CO₂R^{a} C(O)R^{a}, CONR^{a}R^{a}, C₁-C₄-alkyl, C₂-C₄-alkenyl, C₂-C₄-alkynyl and C₁-C₄-haloalkyl; wherein R^{a} is independently at each occurrence selected from H and C₁-C₄ alkyl; and R^{b} is independently at each occurrence selected from H, C₃-C₆-cycloalkyl, C₁-C₄-alkyl, C(O)—C₁-C₄-alkyl and S(O)₂—C₁-C₄-alkyl;

or an agronomically acceptable salt or N-oxide thereof.

In an embodiment, the compound of formula (I) is a compound of formula (II):

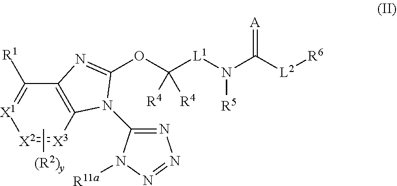

wherein X¹, X², X³, R¹, R², R⁴, R⁵, R⁶, L¹, L², A and y are as described above for compounds of formula (I); and wherein R^{11a} is independently selected from: C₁-C₄-alkyl, C₃-C₄-alkenyl, C₃-C₄-alkynyl, phenyl, C₃-C₆-cycloalkyl, 3- to 6-membered heterocycloalkyl, 5- or 6-membered heteroaryl and C₁-C₄-haloalkyl.

In an embodiment, the compound of formula (I) is a compound of formula (III):

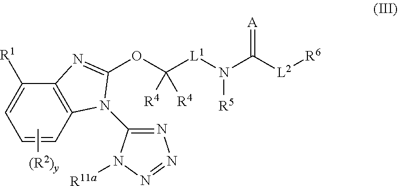

wherein R¹, R², R⁴, R⁵, R⁶, L¹, L², A and y are as described above for compounds of formula (I); and wherein R^{11a} is independently selected from: C₁-C₄-alkyl, C₃-C₄-alkenyl, C₃-C₄-alkynyl, phenyl, C₃-C₆-cycloalkyl, 3- to 6-membered heterocycloalkyl, 5- or 6-membered heteroaryl and C₁-C₄-haloalkyl.

In an embodiment, the compound of formula I is a compound of formula (IV):

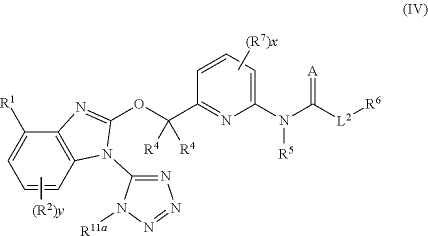

wherein R¹, R², R⁴, R⁵, R⁶, R⁷, L², A and y are as described above for compounds of formula (I); and wherein R^{11a} is independently selected from: C₁-C₄-alkyl, C₃-C₄-alkenyl, C₃-C₄-alkynyl, phenyl, C₃-C₆-cycloalkyl, 3- to 6-membered heterocycloalkyl, 5- or 6-membered heteroaryl and C₁-C₄-haloalkyl; and x is an integer independently selected from 0, 1, 2 and 3.

In an embodiment, the compound of formula (I) is a compound of formula (V):

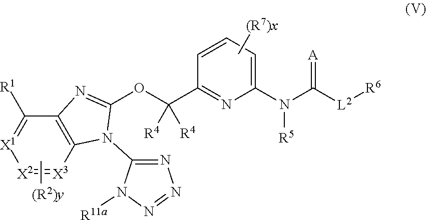

wherein X¹, X², X³, R¹, R², R⁴, R⁵, R⁶, R⁷, L², A and y are as described above for compounds of formula (I); and wherein R^{11a} is independently selected from: C₁-C₄-alkyl, C₃-C₄-alkenyl, C₃-C₄-alkynyl, phenyl, C₃-C₆-cycloalkyl, 3- to 6-membered heterocycloalkyl, 5- or 6-membered heteroaryl and C₁-C₄-haloalkyl; and x is an integer independently selected from 0, 1, 2 and 3.

In an embodiment, the compound of formula (I) is a compound of formula (VI):

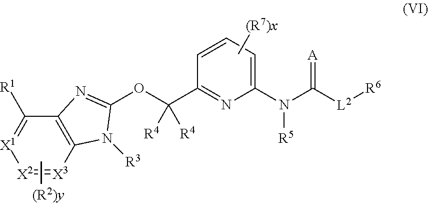

wherein X¹, X², X³, R¹, R², R³, R⁴, R, R⁶, R, L², A and y are as described above for compounds of formula (I); and wherein x is an integer independently selected from 0, 1, 2 and 3.

In an embodiment, the compound of formula (I) is a compound of formula (VII):

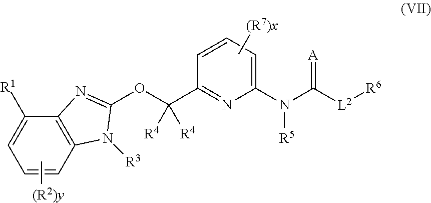

wherein R¹, R², R³, R⁴, R⁵, R⁶, R⁷, L², A and y are as described above for compounds of formula (I); wherein x is an integer independently selected from 0, 1, 2 and 3.

In an embodiment, the compound of formula I is a compound of formula (VII):

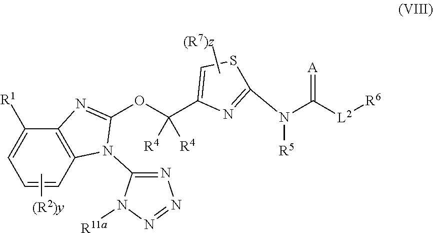

wherein R¹, R², R⁴, R⁵, R⁶, R⁷, L², A and y are as described above for compounds of formula (I); and wherein R^{11a} is independently selected from: C₁-C₄-alkyl, C₃-C₄-alkenyl, C₃-C₄-alkynyl, phenyl, C₃-C₆-cycloalkyl, 3- to 6-membered heterocycloalkyl, 5- or 6-membered heteroaryl and C₁-C₄-haloalkyl; and z is an integer independently selected from 0 and 1.

In an embodiment, the compound of formula (I) is a compound of formula (IX):

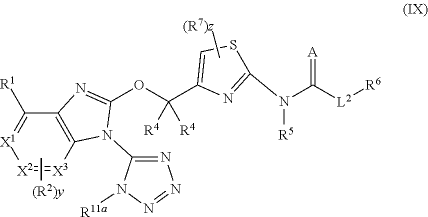

wherein X¹, X², X³, R¹, R², R⁴, R⁵, R⁶, R⁷, L², A and y are as described above for compounds of formula (I); and wherein R^{11a} is independently selected from: C₁-C₄-alkyl, C₃-C₄-alkenyl, C₃-C₄-alkynyl, phenyl, C₃-C₆-cycloalkyl, 3- to 6-membered heterocycloalkyl, 5- or 6-membered heteroaryl and C₁-C₄-haloalkyl; and z is an integer independently selected from 0 and 1.

In an embodiment, the compound of formula (I) is a compound of formula (X):

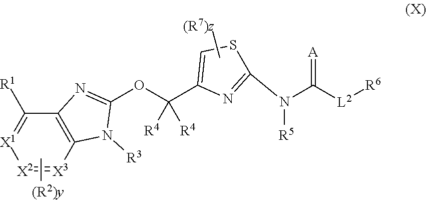

wherein X¹, X², X³, R¹, R², R³, R⁴, R⁵, R⁶, R⁷, L², A and y are as described above for compounds of formula (I); and wherein z is an integer independently selected from 0 and 1.

In an embodiment, the compound of formula (I) is a compound of formula (XI):

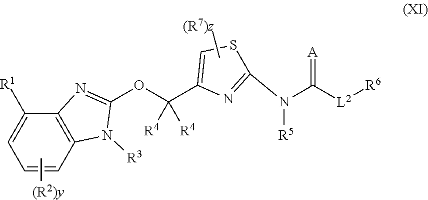

wherein R¹, R², R³, R⁴, R⁵, R⁶, R⁷, L², A and y are as described above for compounds of formula (I); wherein z is an integer independently selected from 0 and 1.

The following embodiments apply to compounds of any of formulae (I)-(XI). These embodiments are independent and interchangeable. Any one embodiment may be combined with any other embodiment, where chemically allowed. In other words, any of the features described in the following embodiments may (where chemically allowable) be combined with the features described in one or more other embodiments. In particular, where a compound is exemplified or illustrated in this specification, any two or more of the embodiments listed below, expressed at any level of generality, which encompass that compound may be combined to provide a further embodiment which forms part of the present disclosure.

X¹ may be nitrogen. X¹ may be carbon. X² may be nitrogen. X² may be carbon. X³ may be nitrogen. X³ may be carbon. It may be that no more than two of X¹, X² and X³ are nitrogen. It may be that no more than one of X¹, X² and X³ are nitrogen. It may be that a single one of X¹, X² and X³ is nitrogen. It may be that each of X¹, X² and X³ are carbon.

L¹ may be a 5- or 6-membered heteroaryl group having a nitrogen atom in the ring adjacent to the carbon atom in the ring through which L¹ is connected to the carbon having two R⁴ groups.

L¹ may be a 6-membered heteroaryl group, e.g. a 6-membered heteroaryl group having a nitrogen atom in the ring adjacent to the carbon atom in the ring through which L¹ is connected to the carbon having two R⁴ groups. L¹ may be a 6-membered heteroaryl group having two nitrogen atoms in the ring, e.g. a 6-membered heteroaryl group having two nitrogen atoms in the ring, wherein one nitrogen atom in the ring is adjacent to the carbon atom in the ring through which L¹ is connected to the carbon having two R⁴ groups. L¹ may be pyridine, pyridazine or pyrazine. Alternatively, L¹ may be pyridine, e.g. a pyridine group wherein the nitrogen atom in the ring is adjacent to the carbon having two R⁴ groups.

L¹ may be a 5-membered heteroaryl group comprising at least one nitrogen atom, e.g. a 5-membered heteroaryl group having a nitrogen atom in the ring adjacent to the carbon through which L¹ is connected to the carbon having two R⁴ groups. L¹ may be a thiazole group e.g. a thiazole group having a nitrogen atom in the ring adjacent to the carbon through which L¹ is connected to the carbon having two R⁴ groups.

In embodiments in which a nitrogen is adjacent to the carbon having two R⁴ groups, the group NR⁵C(=A)L²R⁶ is attached to a second carbon that is also adjacent to said nitrogen.

R⁷ may be independently at each occurrence selected from halo, nitro, cyano, NR⁸R⁹, NR⁸CONR⁸R⁸, NR⁸CO₂R⁸, OR¹⁰, SR⁸, S(O)R⁸, S(O)₂R⁸, OS(O)₂OR⁸, S(O)₂NR⁸R⁸, CO₂R⁸, C(O)R⁸, CONR⁸R⁸, C₁-C₄-alkyl, C₂-C₄-alkenyl, C₂-C₄-alkynyl, and C₁-C₄-haloalkyl. R⁷ may be independently at each occurrence selected from halo, cyano, C₁-C₄-alkyl and C₁-C₄-haloalkyl.

R⁷ may be independently at each occurrence selected from: halo, cyano, C₁-C₄-alkyl, C₂-C₄-alkenyl, C₂-C₄-alkynyl, phenyl, C₃-C₆-cycloalkyl, 3- to 6-membered heterocycloalkyl, 5- or 6-membered heteroaryl and C₁-C₄-haloalkyl.

x may be an integer from 0 to 2. x may be an integer from 1 to 3, e.g. from 1 to 2. x may be 1. x may be 0.

z may be 0.

R⁵ may be C₁-C₄-alkyl, e.g. Me or Et. Preferably, however, R⁵ is H.

=A may be ═S. Preferably, =A is ═O.

-L²- may be absent. In these embodiments, R⁶ may be independently selected from: C₁-C₈-alkyl, C₂-C₈-alkenyl, C₂-C₈-alkynyl and C₀-C₃-alkylene-R¹². In these embodiments, R⁶ may be independently selected from: C₁-C₈-alkyl, C₁-C₈-haloalkyl, and C₀-C₃-alkylene-R¹². R¹² may be selected from C₃-C₆-cycloalkyl, 6-membered heteroaryl and phenyl. R¹² may be phenyl. R¹² may be C₃-C₆-cycloalkyl. R¹² may be pyridyl. R⁶ may be independently selected from: C₁-C₈-alkyl and C₀-C₃-alkylene-R¹², where R¹² is selected from phenyl and C₃-C₆-cycloalkyl. R⁶ may be independently selected from: C₁-C₈-alkyl and C₀-C₃-alkylene-R¹², where R¹² is C₃-C₆-cycloalkyl. Said R⁶ and R¹² groups may be unsubstituted.

Illustrative NR⁵C(=A)L²R⁶ groups include:

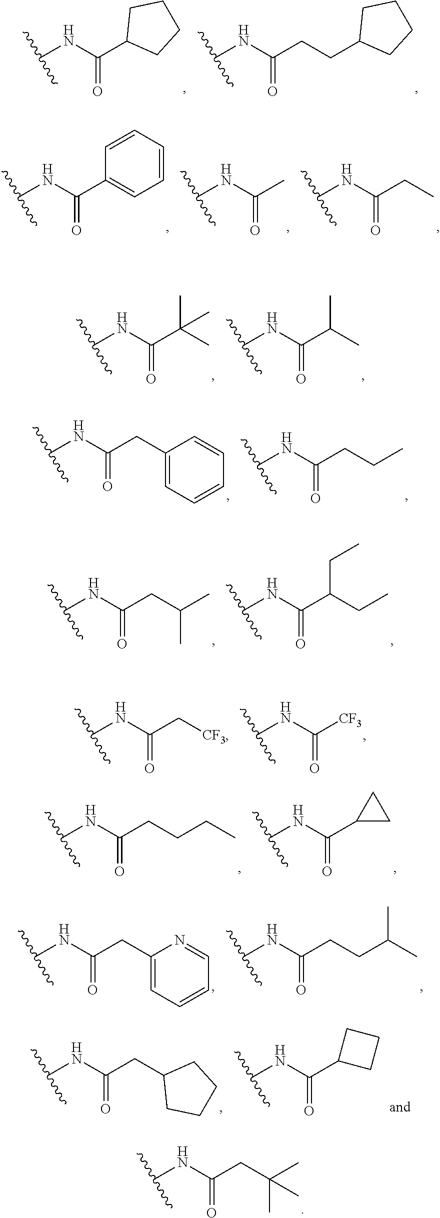

-L²- may be absent. In these embodiments, R⁶ may be independently selected from: 3- to 6-membered heterocycloalkyl, 5- or 6-membered heteroaryl and —CR¹³R¹³L³R¹⁴. R⁶ may be independently selected from: 3- to 6-membered heterocycloalkyl, 5- or 6-membered heteroaryl. R may be —CR¹³R¹³L³R¹⁴.

R¹³ is preferably at all occurrences independently selected from F, H and Me. R¹³ may at all occurrences be selected from F and H. R¹³ may at all occurrences be H. R¹³ may at all occurrences be F.

-L³- may be —NR¹⁷—, e.g. NH. -L³- may be —S—. -L³- may be —O—.

R⁶ may be CR¹³R¹³OR¹⁴ or CR¹³R¹³SR¹⁴, where R¹⁴ is independently selected from C₁-C₃-alkyl, C₁-C₃-haloalkyl, C₃-C₃-alkenyl, C₃-C₃-alkynyl and C₀-C₃-alkylene-R¹⁶; wherein R¹⁶ is independently at each occurrence selected from C₃-C₆-cycloalkyl, 3- to 6-membered heterocycloalkyl, 5- or 6-membered heteroaryl and phenyl. R¹⁴ may be independently selected from: C₁-C₃-alkyl, C₃-C₃-alkenyl, C₃-C₃-alkynyl and C₀-C₃-alkylene-R¹⁶. R¹⁶ may be selected from C₃-C₆-cycloalkyl and phenyl. R¹⁶ may be phenyl. R¹⁶ may be C₃-C₆-cycloalkyl. R¹⁴ may be independently selected from: C₁-C₆-alkyl and C₀-C₃-alkylene-R¹⁶, where R¹⁶ is selected from phenyl and C₃-C₆-cycloalkyl. Said R¹⁴ and R¹⁶ groups may be unsubstituted. R⁶ may be CR¹³R¹³OR¹⁶, where R¹⁶ is independently selected from: unsubstituted C₁-C₃-alkyl, unsubstituted C₃-C₆-cycloalkyl and unsubstituted phenyl.

Illustrative NR⁵C(=A)L²R⁶ groups include:

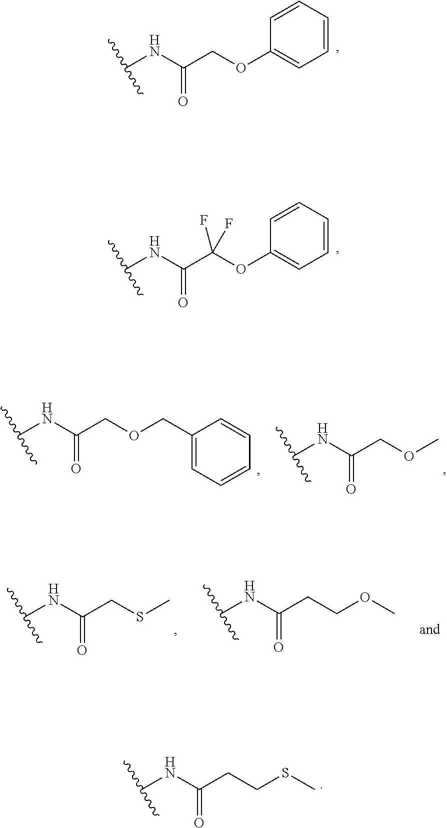

-L²- may be independently selected from: —O—, —S— and —NR¹⁶—. -L²- may be —O—. In these embodiments, R⁶ may be independently selected from: C₁-C₃-alkyl, C₃-C₃-alkenyl, C₃-C₃-alkynyl and C₀-C₃-alkylene-R¹². R¹² may be selected from C₃-C₆-cycloalkyl and phenyl. R¹² may be phenyl. R¹² may be C₃-C₆-cycloalkyl. R⁶ may be independently selected from: C₁-C₃-alkyl and C₀-C₃-alkylene-R¹², where R¹² is selected from phenyl and C₃-C₆-cycloalkyl. R⁶ may be independently selected from: C₁-C₃-alkyl and C₀-C₃-alkylene-R¹², where R¹² is C₃-C₆-cycloalkyl. Said R⁶ and R¹² groups may be unsubstituted. R⁶ may be C₁-C₃-alkyl. R⁶ may be C₃-C₃-alkyl.

Illustrative NR⁵C(=A)L²R⁶ groups include:

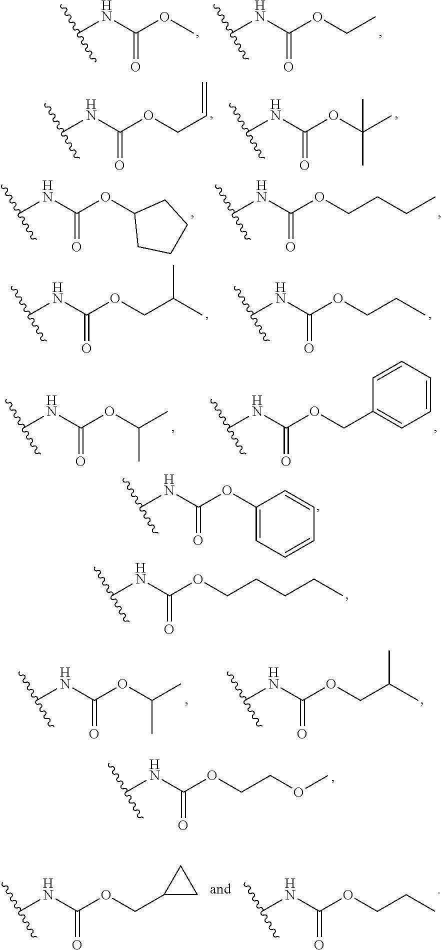

It may be that R⁴ is independently at each occurrence selected from H, F, C₁-C₄-alkyl and C₁-C₄-haloalkyl. It may be that R⁴ is independently at each occurrence selected from H, F, Me, CF₃ and Et. It may be that R⁴ is independently at each occurrence selected from H and Me. It may be that R⁴ is at each occurrence H.

It may be that when R⁶ is —CR¹³R¹³L³R¹⁴-L²- is absent.

R³ may be a 5- or 6-membered heteroaryl group having 1, 2, 3 or 4 nitrogen atoms in the ring. R³ may be a 5-membered heteroaryl group having 1, 2, 3 or 4 nitrogen atoms in the ring. R³ may be a 6-membered heteroaryl group having 1 or 2 nitrogen atoms in the ring.

R³ may be substituted at a position adjacent to the point of connection of R³ to the rest of the molecule with an R^{11b} group, wherein R^{11b} is selected from C₁-C₄-alkyl, C₂-C₄-alkenyl, C₂-C₄-alkynyl, phenyl, C₃-C₆-cycloalkyl, 3- to 6-membered heterocycloalkyl, 5- or 6-membered heteroaryl and C₁-C₄-haloalkyl. R³ may be a 5- or 6-membered heteroaryl group having 1, 2, 3 or 4 nitrogen atoms in the ring, said heteroaryl group being substituted at a position adjacent to the point of connection of R³ to the rest of the molecule with an R^{11b} group. R³ may be a 5-membered heteroaryl group having 1, 2, 3 or 4 nitrogen atoms in the ring, said heteroaryl group being substituted at a position adjacent to the point of connection of R³ to the rest of the molecule with an R^{11b} group. R³ may be a 6-membered heteroaryl group having 1 or 2 nitrogen atoms in the ring, said heteroaryl group being substituted at a position ortho to the point of connection of R³ to the rest of the molecule with an R^{11b} group.

R³ may be a tetrazole ring. Said tetrazole ring is substituted with a single R^{11a} group; wherein R^{11a} is independently selected from: C₁-C₄-alkyl, C₃-C₄-alkenyl, C₃-C₄-alkynyl, phenyl, C₃-C₆-cycloalkyl, 3- to 6-membered heterocycloalkyl, 5- or 6-membered heteroaryl and C₁-C₄-haloalkyl. Said tetrazole will typically be attached to the rest of the molecule via the carbon atom of the tetrazole ring. R^{11a} may be attached to a nitrogen atom neighbouring said carbon atom. Thus, R³ may be:

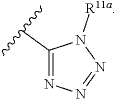

R^{11a} may be selected from C₁-C₄-alkyl, C₃-C₄-alkenyl, C₃-C₄-alkynyl, phenyl, C₃-C₆-cycloalkyl, 3- to 6-membered heterocycloalkyl, 5- or 6-membered heteroaryl and C₁-C₄-haloalkyl. R^{11a} may be selected from C₁-C₄-alkyl, C₁-C₄-haloalkyl and C₃-C₆-cycloalkyl. R^{11a} may be C₁-C₄-alkyl, e.g. methyl.

Thus, R³ may be:

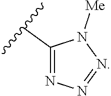 [SMILES: *c1nnnn1C]

R³ may be selected from isoxazole, pyrazole or isothiazole. Thus, R³ may be:

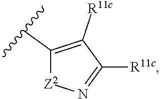

where Z² is selected from O, S and NR^{11a}; wherein R^{11a} is independently selected from: H, C₁-C₄-alkyl, C₃-C₄-alkenyl, C₃-C₄-alkynyl, phenyl, C₃-C₆-cycloalkyl, 3- to 6-membered heterocycloalkyl, 5- or 6-membered heteroaryl and C₁-C₄-haloalkyl; and wherein R^{11c} is selected from H and R¹¹.

R³ may be:

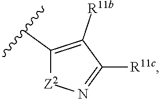

wherein R^{11b} is selected from C₁-C₄-alkyl, C₂-C₄-alkenyl, C₂-C₄-alkynyl, phenyl, C₃-C₆-cycloalkyl, 3- to 6-membered heterocycloalkyl, 5- or 6-membered heteroaryl and C₁-C₄-haloalkyl; and wherein R¹ is selected from H and R¹¹. Z² may be S. Z² may be O. Z² may be NR^{11a}.

Alternatively, R³ may be:

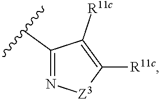

where Z³ is selected from O, S and NR^{11a}; wherein R^{11a} is independently selected from: H, C₁-C₄-alkyl, C₃-C₄-alkenyl, C₃-C₄-alkynyl, phenyl, C₃-C₆-cycloalkyl, 3- to 6-membered heterocycloalkyl, 5- or 6-membered heteroaryl and C₁-C₄-haloalkyl; and wherein R^{11c} is selected from H and R¹¹.

R³ may be:

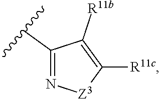

wherein R^{11b} is selected from C₁-C₄-alkyl, C₂-C₄-alkenyl, C₂-C₄-alkynyl, phenyl, C₃-C₆-cycloalkyl, 3- to 6-membered heterocycloalkyl, 5- or 6-membered heteroaryl and C₁-C₄-haloalkyl; and wherein R^{11c} is selected from H and R¹¹. Z³ may be S. Z³ may be O. Z³ may be NR^{11a}.

Illustrative examples of R³ include:

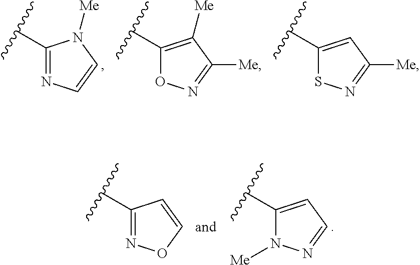

R³ may be a 6-membered heteroaromatic ring. R³ may be a pyridine. R³ may be a 2-pyridine. R³ may be a pyrazine. R³ may be a pyridazine.

R³ may be:

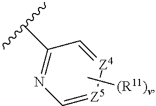

wherein Z⁴ and Z⁵ are each independently selected from nitrogen or carbon; and v is an integer from 0 to 4. For the absence of doubt, where Z⁴ and/or Z⁵ is carbon, said carbon may be substituted with an R¹¹ group.

R³ may be

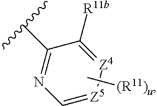

wherein R^{11b} is selected from C₁-C₄-alkyl, C₂-C₄-alkenyl, C₂-C₄-alkynyl, phenyl, C₃-C₆-cycloalkyl, 3- to 6-membered heterocycloalkyl, 5- or 6-membered heteroaryl and C₁-C₄-haloalkyl; and wherein u is an integer from 0 to 3.

Z⁴ may be carbon. Z⁴ may be N.

Z⁵ may be carbon. Z⁵ may be N. It may be that a single one of Z⁴ and Z⁵ is nitrogen. It may be that Z⁴ and Z⁵ are each carbon. It may be that Z⁴ is carbon and Z⁵ is nitrogen. It may be that Z⁵ is carbon and Z⁴ is nitrogen.

Illustrative examples of R³ include:

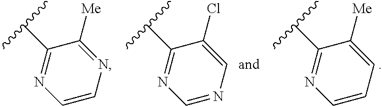 [SMILES: *c1nccnc1C ; *c1ncncc1Cl ; "and" ; *c1ncccc1C ; "."]

R^{11a} may be independently selected from: H, C₁-C₄-alkyl, C₃-C₄-alkenyl and C₃-C₄-alkynyl. R^{11a} may be independently selected from: H and C₁-C₄-alkyl. R^{11a} may be independently selected from: C₁-C₄-alkyl, C₃-C₄-alkenyl and C₃-C₄-alkynyl. R^{11a} may be H. R^{11a} may be C₁-C₄-alkyl, e.g. methyl.

R^{11b} may be selected from C₁-C₄-alkyl, C₁-C₄-haloalkyl and C₃-C₆-cycloalkyl. R^{11b} may be C₁-C₄-alkyl, e.g. methyl.

R^{11c} may be at all occurrences H.

v may be 1 or 2. u may be 1 or 2. v may be 0. u may be 0.

R¹¹ is independently at each occurrence selected from: halo, nitro, cyano, NR⁸R⁹, NR⁸CONR⁸R⁸, OR¹⁰, SR⁸, S(O)R⁸, S(O)₂R⁸, S(O)₂NR⁸R⁸, OS(O)₂OR⁸, CO₂R⁸, C(O)R⁸, CONR⁸R⁸, C₁-C₄-alkyl, C₂-C₄-alkenyl, C₂-C₄-alkynyl, phenyl, C₃-C₆-cycloalkyl, 3- to 6-membered heterocycloalkyl, 5- or 6-membered heteroaryl, C₁-C₄-haloalkyl and —O—C₁-C₄-haloalkyl. R¹¹ may be independently at each occurrence selected from halo, nitro, cyano, NR⁸R⁹, NR⁸CONR⁸R⁸, OR¹⁰, SR⁸, S(O)R⁸, S(O)₂R⁸, S(O)₂NR⁸R⁸, OS(O)₂OR⁸, CO₂R⁸, C(O)R⁸, CONR⁸R⁸, C₁-C₄-alkyl, C₂-C₄-alkenyl, C₂-C₄-alkynyl, C₁-C₄-haloalkyl and —O—C₁-C₄-haloalkyl. R¹¹ may be independently at each occurrence selected from halo, cyano, C₁-C₄-alkyl and C₁-C₄-haloalkyl. R¹¹ may be independently at each occurrence selected from: halo, cyano, C₁-C₄-alkyl, C₂-C₄-alkenyl, C₂-C₄-alkynyl, phenyl, C₃-C₆-cycloalkyl, 3- to 6-membered heterocycloalkyl, 5- or 6-membered heteroaryl and C₁-C₄-haloalkyl.

y may be 0. Alternatively, y may be 1 or 2. R² may be independently at each occurrence selected from halo, cyano, C₁-C₄-alkyl and C₁-C₄-haloalkyl and —O—C₁-C₄-haloalkyl.

R¹ may be selected from chloro, C₁-C₄-alkyl and OR¹⁰. R¹ may be selected from chloro, C₁-C₄-alkyl and OR¹⁰; wherein R¹⁰ is selected from H, C₁-C₄-alkyl, C₁-C₄-alkenyl, C₁-C₄-alkynyl and C₁-C₄-haloalkyl. It may be that R¹ may be selected from chloro, bromo, C₁-C₄-alkyl and C₁-C₄-haloalkyl, O—C₁-C₄-alkyl and O—C₁-C₄-haloalkyl. R¹ may be chloro or bromo. R¹ may be chloro. R¹ may be bromo.

It may be that R¹⁰ is independently at each occurrence selected from H, C₁-C₆-alkyl, C₁-C₆-haloalkyl, and C₃-C₆-cycloalkyl.

The compound of formula (I) may be a compound selected from:

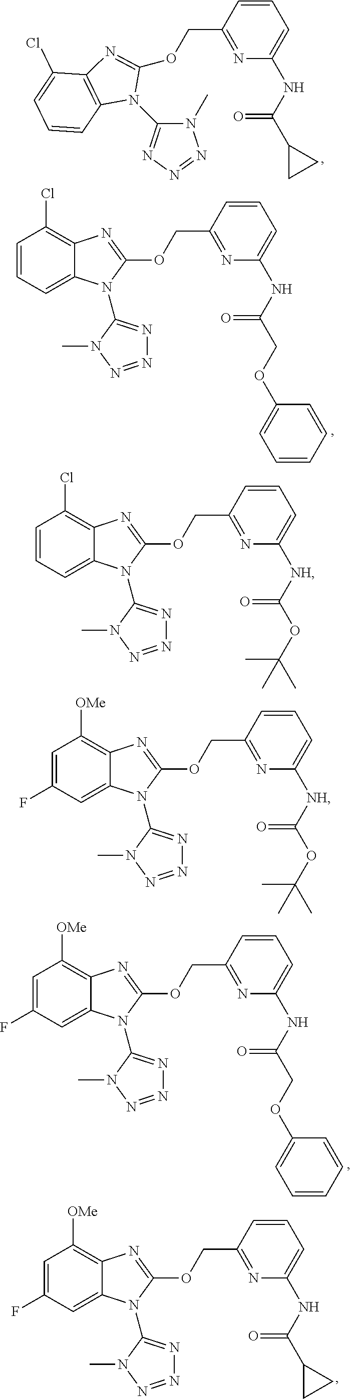

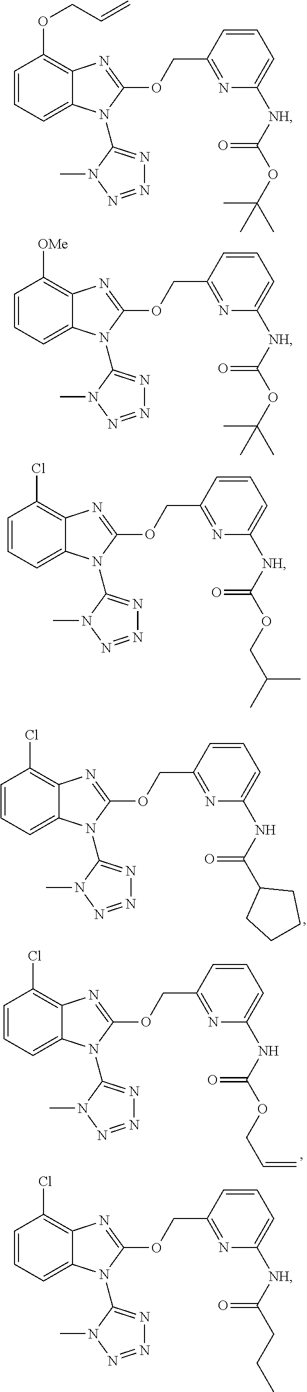

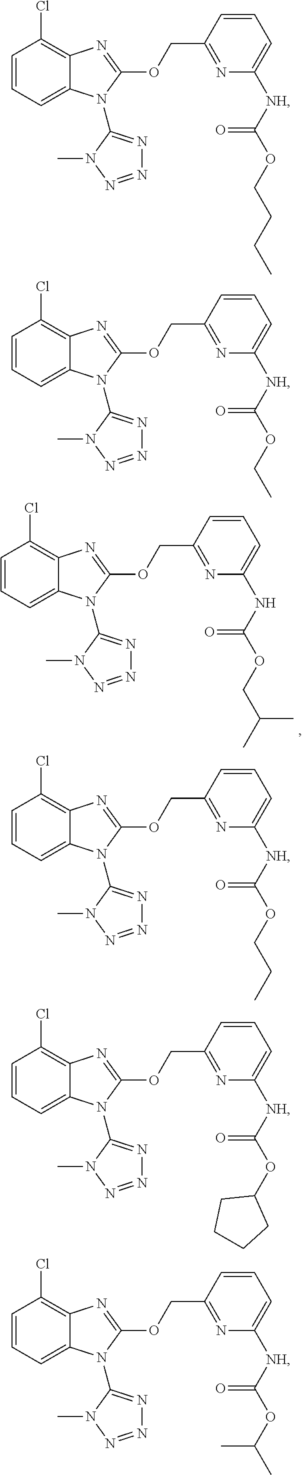

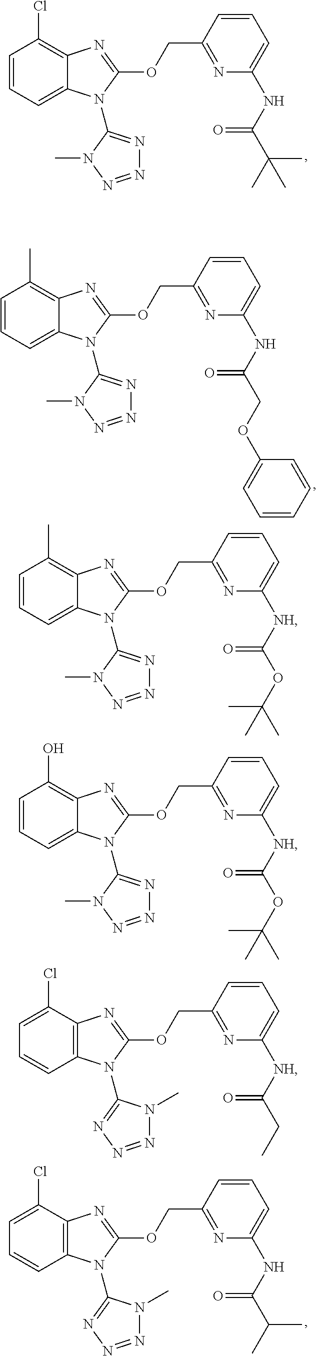

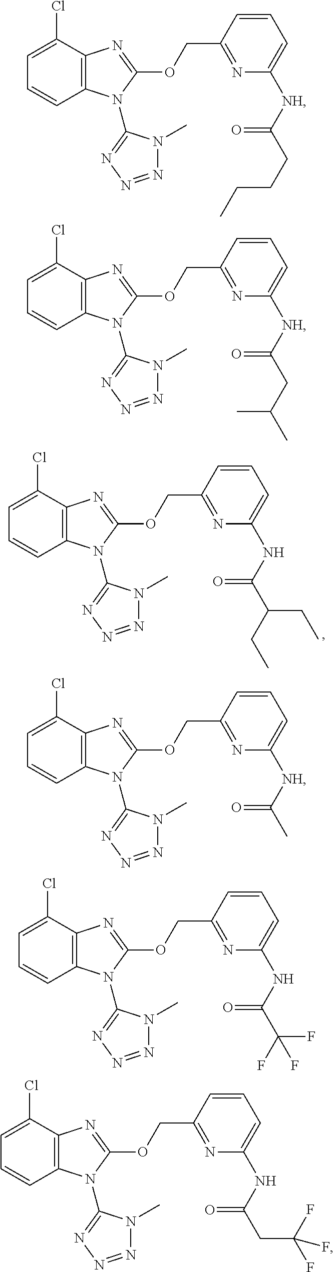

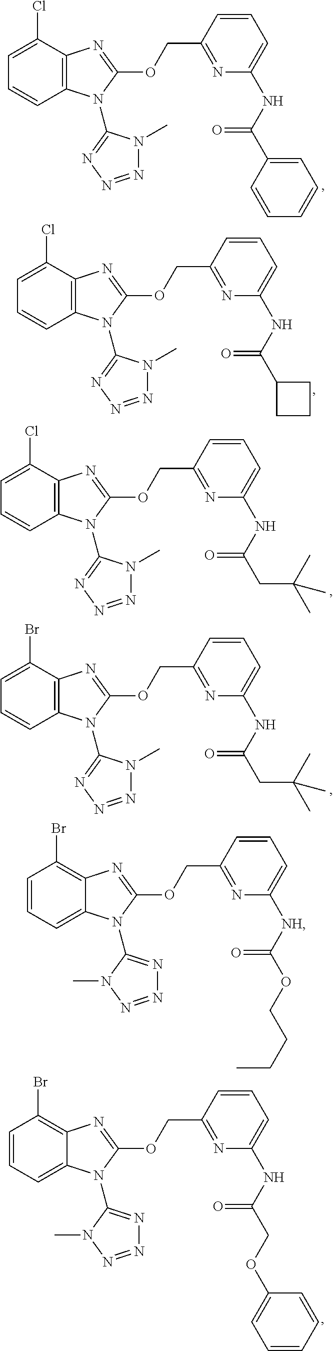

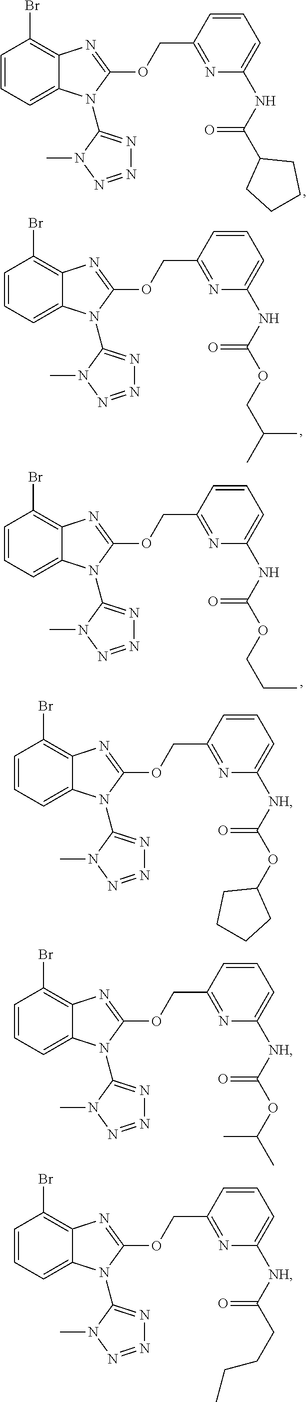

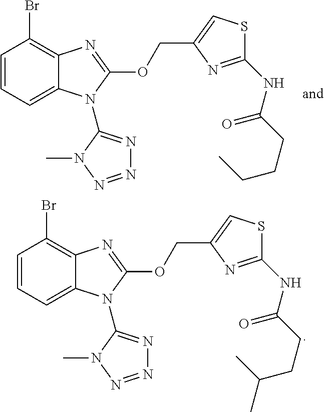

### DETAILED DESCRIPTION

The term Cₘ-Cₙ refers to a group with m to n carbon atoms.

The term “alkyl” refers to a monovalent linear or branched saturated hydrocarbon chain. For example, “C₁-C₆-alkyl” may refer to methyl, ethyl, n-propyl, iso-propyl, n-butyl, sec-butyl, tert-butyl, n-pentyl and n-hexyl. The alkyl groups may be unsubstituted or substituted by one or more substituents. Specific substituents for each alkyl group independently may be fluorine, OR^{a} or NHR^{a}.

The term “alkylene” refers to a bivalent linear saturated hydrocarbon chain. For example, “C₁-C₃-alkylene” may refer to methylene, ethylene or propylene. The alkylene groups may be unsubstituted or substituted by one or more substituents. Specific substituents for each alkylene group independently may be methyl, fluorine, OR^{a} or NHR^{a}.

The term “haloalkyl” refers to a hydrocarbon chain substituted with at least one halogen atom independently chosen at each occurrence from: fluorine, chlorine, bromine and iodine. The halogen atom may be present at any position on the hydrocarbon chain. For example, “C₁-C₆-haloalkyl” may refer to chloromethyl, fluoromethyl, trifluoromethyl, chloroethyl e.g. 1-chloromethyl and 2-chloroethyl, trichloroethyl e.g. 1,2,2-trichloroethyl, 2,2,2-trichloroethyl, fluoroethyl e.g. 1-fluoroethyl and 2-fluoroethyl, trifluoroethyl e.g. 1,2,2-trifluoroethyl and 2,2,2-trifluoroethyl, chloropropyl, trichloropropyl, fluoropropyl, trifluoropropyl. A haloalkyl group may be a fluoroalkyl group, i.e. a hydrocarbon chain substituted with at least one fluorine atom. Thus, a haloalkyl group may have any amount of halogen substituents. The group may contain a single halogen substituent, it may have two or three halogen substituents, or it may be saturated with halogen substituents.

The term “alkenyl” refers to a branched or linear hydrocarbon chain containing at least one double bond. The double bond(s) may be present as the E or Z isomer. The double bond may be at any possible position of the hydrocarbon chain. For example, “C₂-C₆-alkenyl” may refer to ethenyl, propenyl, butenyl, butadienyl, pentenyl, pentadienyl, hexenyl and hexadienyl. The alkenyl groups may be unsubstituted or substituted by one or more substituents. Specific substituents for any saturated carbon atom in each alkenyl group independently may be fluorine, OR^{a} or NHR^{a}.

The term “alkynyl” refers to a branched or linear hydrocarbon chain containing at least one triple bond. The triple bond may be at any possible position of the hydrocarbon chain. For example, “C₂-C₆-alkynyl” may refer to ethynyl, propynyl, butynyl, pentynyl and hexynyl. The alkynyl groups may be unsubstituted or substituted by one or more substituents. Specific substituents for any saturated carbon atom in each alkynyl group independently may be fluorine, OR^{a} or NHR^{a}.

The term “cycloalkyl” refers to a saturated hydrocarbon ring system containing 3, 4, 5 or 6 carbon atoms. For example, “C₃-C₆-cycloalkyl” may refer to cyclopropyl, cyclobutyl, cyclopentyl, cyclohexyl. The cycloalkyl groups may be unsubstituted or substituted by one or more substituents. Specific substituents for each cycloalkyl group independently may be fluorine, OR^{a} or NHR^{a}.

The term “y- to z-membered heterocycloalkyl group” may refer to a monocyclic or bicyclic saturated or partially saturated group having from y to z atoms in the ring system and comprising 1 or 2 heteroatoms independently selected from O, S and N in the ring system (in other words 1 or 2 of the atoms forming the ring system are selected from O, S and N). By partially saturated it is meant that the ring may comprise one or two double bonds. This applies particularly to monocyclic rings with from 5 to 6 members. The double bond will typically be between two carbon atoms but may be between a carbon atom and a nitrogen atom. A heterocycloalkyl group may mean a saturated heterocycloalkyl group. Examples of heterocycloalkyl groups include; piperidine, piperazine, morpholine, thiomorpholine, pyrrolidine, tetrahydrofuran, tetrahydrothiophene, dihydrofuran, tetrahydropyran, dihydropyran, dioxane, azepine. A heterocycloalkyl group may be unsubstituted or substituted by one or more substituents. Specific substituents for any saturated carbon atom in each heterocycloalkyl group may independently be fluorine, OR^{a} or NHR^{a}.

The term “oxo” refers to an oxygen atom attached via a double bonded to a carbon atom in the indicated group.

Aryl groups may be any aromatic carbocyclic ring system (i.e. a ring system containing 2(2n+1)π electrons). Aryl groups may have from 6 to 12 carbon atoms in the ring system. Aryl groups will typically be phenyl groups. Aryl groups may be naphthyl groups or biphenyl groups.

In any of the above aspects and embodiments, heteroaryl groups may be any aromatic (i.e. a ring system containing 2(2n+1)π electrons) 5 or 6 membered ring system comprising from 1 to 4 heteroatoms independently selected from O, S and N (in other words from 1 to 4 of the atoms forming the ring system are selected from O, S and N). Thus, any heteroaryl groups may be independently selected from: 5-membered heteroaryl groups in which the heteroaromatic ring is substituted with 1-4 heteroatoms independently selected from 0, S and N; and 6-membered heteroaryl groups in which the heteroaromatic ring is substituted with 1-3 (e.g. 1-2) nitrogen atoms. Specifically, heteroaryl groups may be independently selected from: pyrrole, furan, thiophene, pyrazole, imidazole, oxazole, isoxazole, triazole, oxadiazole, thiadiazole, tetrazole; pyridine, pyridazine, pyrimidine, pyrazine, triazine.

It may be that, in any group which is an aryl or heteroaryl group, that aryl or heteroaryl group may be unsubstituted or is optionally substituted, where chemically possible, by 1 to 5 substituents which are each independently selected at each occurrence from: halo, nitro, cyano, NR^{a}R^{a}, NR^{a}S(O)₂R^{a}, NR^{a}C(O)R^{a}, NR^{a}CONR^{a}R^{a}, NR^{a}CO₂R^{a}, OR^{a}, SR^{a}, S(O)R^{a}, S(O)₂R^{a}, S(O)₂NR^{a}R^{a}, CO₂R^{a} C(O)R^{a}, CONR^{a}R^{a}, CR^{b}R^{b}NR^{a}R^{a}, CR^{b}R^{b}OR^{a}, C₁-C₄-alkyl, C₂-C₄-alkenyl, C₂-C₄-alkynyl and C₁-C₄-haloalkyl; wherein R^{a} is as described above for formula I.

Compounds of the invention containing one or more asymmetric carbon atoms can exist as two or more stereoisomers. Where a compound of the invention contains a double bond such as a C═C or C═N group, geometric cis/trans (or Z/E) isomers are possible. Where structural isomers are interconvertible via a low energy barrier, tautomeric isomerism (‘tautomerism’) can occur. This can take the form of proton tautomerism in compounds of the invention containing, for example, an imino, keto, or oxime group, or so-called valence tautomerism in compounds which contain an aromatic moiety. It follows that a single compound may exhibit more than one type of isomerism.

Included within the scope of the present invention are all stereoisomers, geometric isomers and tautomeric forms of the compounds of the invention, including compounds exhibiting more than one type of isomerism, and mixtures of one or more thereof. Also included are acid addition or base salts wherein the counter ion is optically active, for example, d-lactate or I-lysine, or racemic, for example, dl-tartrate or dl-arginine.

The compounds of the invention may be obtained, stored and/or used in the form of an agronomically acceptable salt. Suitable salts include, but are not limited to, salts of acceptable inorganic acids such as hydrochloric, sulfuric, phosphoric, nitric, carbonic, boric, sulfamic, and hydrobromic acids, or salts of agronomically acceptable organic acids such as acetic, propionic, butyric, tartaric, maleic, hydroxymaleic, fumaric, malic, citric, lactic, mucic, gluconic, benzoic, succinic, oxalic, phenylacetic, methanesulfonic, toluenesulfonic, benzenesulfonic, salicylic, sulfanilic, aspartic, glutamic, edetic, stearic, palmitic, oleic, lauric, pantothenic, tannic, ascorbic and valeric acids. Suitable salts also include salts of inorganic and organic bases, e.g. counterions such as Na, Ca, K, Li, Mg, ammonium, trimethylsulfonium. The compounds may also be obtained, stored and/or used in the form of an N-oxide.

Cis/trans isomers may be separated by conventional techniques well known to those skilled in the art, for example, chromatography and fractional crystallisation.

Conventional techniques for the preparation/isolation of individual enantiomers when necessary include chiral synthesis from a suitable optically pure precursor or resolution of the racemate (or the racemate of a salt or derivative) using, for example, chiral high pressure liquid chromatography (HPLC). Thus, chiral compounds of the invention (and chiral precursors thereof) may be obtained in enantiomerically-enriched form using chromatography, typically HPLC, on an asymmetric resin with a mobile phase consisting of a hydrocarbon, typically heptane or hexane, containing from 0 to 50% by volume of isopropanol, typically from 2% to 20%, and for specific examples, 0 to 5% by volume of an alkylamine e.g. 0.1% diethylamine. Concentration of the eluate affords the enriched mixture.

Alternatively, the racemate (or a racemic precursor) may be reacted with a suitable optically active compound, for example, an alcohol, or, in the case where the compound of the invention contains an acidic or basic moiety, a base or acid such as 1-phenylethylamine or tartaric acid. The resulting diastereomeric mixture may be separated by chromatography and/or fractional crystallization and one or both of the diastereoisomers converted to the corresponding pure enantiomer(s) by means well known to a skilled person.

When any racemate crystallises, crystals of two different types are possible. The first type is the racemic compound (true racemate) referred to above wherein one homogeneous form of crystal is produced containing both enantiomers in equimolar amounts. The second type is the racemic mixture or conglomerate wherein two forms of crystal are produced in equimolar amounts each comprising a single enantiomer.

While both of the crystal forms present in a racemic mixture have identical physical properties, they may have different physical properties compared to the true racemate. Racemic mixtures may be separated by conventional techniques known to those skilled in the art—see, for example, “Stereochemistry of Organic Compounds” by E. L. Eliel and S. H. Wilen (Wiley, 1994).

The activity of the compounds of the present invention can be assessed by a variety of in silico, in vitro and in vivo assays. In silico analysis of a variety of compounds has been demonstrated to be predictive of ultimate in vitro and even in vivo activity.

The present invention also includes all environmentally acceptable isotopically-labelled compounds of formulae (I) to (XI) and their syntheses, wherein one or more atoms are replaced by atoms having the same atomic number, but an atomic mass or mass number different from the atomic mass or mass number usually found in nature.

Examples of isotopes suitable for inclusion in the compounds of the invention include isotopes of hydrogen, such as ²H and ³H, carbon, such as ¹¹C, ¹³C and ¹⁴C, chlorine, such as ³⁶Cl, fluorine, such as ¹⁸F, iodine, such as ¹²³I and ¹²⁵I, nitrogen, such as ¹³N and ¹⁵N, oxygen, such as ¹⁵O, ¹⁷O and ¹⁸O, phosphorus, such as ³²P, and sulfur, such as ³⁵S.

Isotopically-labelled compounds can generally be prepared by conventional techniques known to those skilled in the art or by processes analogous to those described using an appropriate isotopically-labelled reagent in place of the non-labelled reagent previously employed.

Throughout the description and claims of this specification, the words “comprise” and “contain” and variations of the words, for example “comprising” and “comprises”, means “including but not limited to”, and is not intended to (and does not) exclude other moieties, additives, components, integers or steps.

Throughout the description and claims of this specification, the singular encompasses the plural unless the context otherwise requires. In particular, where the indefinite article is used, the specification is to be understood as contemplating plurality as well as singularity, unless the context requires otherwise.

Features, integers, characteristics, compounds, chemical moieties or groups described in conjunction with a particular aspect, embodiment or example of the invention are to be understood to be applicable to any other aspect, embodiment or example described herein unless incompatible therewith.

If appropriate, the compounds of the invention can, at certain concentrations or application rates, be used as fungicides.

According to another aspect of the present invention, there is provided a method for controlling the fungal diseases of plants, crops or seeds, the method comprising applying an agronomically effective and substantially non-phytotoxic quantity of a compound according to the invention to the seeds of the plants, to the plants themselves or to the area where it is intended that the plants will grow.

The pesticide may be applied as a seed treatment, foliar application, stem application, drench or drip application (chemigation) to the seed, the plant or to the fruit of the plant or to soil or to inert substrate (e.g. inorganic substrates like sand, rockwool, glasswool; expanded minerals like perlite, vermiculite, zeolite or expanded clay), Pumbe, Pyroclastic materials or stuff, synthetic organic substrates (e.g. polyurethane) organic substrates (e.g. peat, composts, tree waste products like coir, wood fibre or chips, tree bark) or to a liquid substrate (e.g. floating hydroponic systems, Nutrient Film Technique, Aeroponics).

In a further aspect, the present invention also relates to a fungicidal composition comprising an effective and non-phytotoxic amount of an active compound of the invention. The composition may further comprise one or more additional fungicides.

The term “effective and non-phytotoxic amount” means an amount of pesticide according to the invention which is sufficient to control or destroy any of the targeted pests present or liable to appear on the crops and which does not have any significant detrimental effect on the crops or indeed has a positive effect on plant vigour and yield in the absence of target organism. The amount will vary depending on the pest to be controlled, the type of crop, the climatic conditions and the compounds included in the pesticidal composition. This amount can be determined by systematic field trials, which are within the capabilities of a person skilled in the art.

Depending on their particular physical and/or chemical properties, the active compounds of the invention can be formulated as solutions, emulsions, suspensions, powders, foams, pastes, granules, aerosols, microencapsulations in polymeric substances and in coating materials for seed, and also as ULV cold and warm fogging formulations.

The active compounds can be used neat, or in the form of a formulation, e.g. ready-to-use solutions, emulsions, water- or oil-based suspensions, powders, wettable powders, pastes, soluble powders, dusts, soluble granules, granules for broadcasting, suspoemulsion concentrates, natural substances impregnated with active compound, synthetic substances impregnated with active compound, fertilizers and also microencapsulations in polymeric substances. Application may be carried out, for example, by watering, spraying, atomizing, broadcasting, dusting, foaming, spreading, etc. It is also possible to apply the active compounds by the ultra-low volume method or to inject the preparation of active compound or the active compound itself into the soil. It is also possible to treat the seed of the plants.

Formulations containing the compounds of the invention are produced in a known manner, for example by mixing the compounds with extenders (e.g. liquid solvents and/or solid carriers), optionally with the use of surfactants (e.g. emulsifiers and/or dispersants and/or foam-formers). The formulations are prepared either in factories/production plants or alternatively before or during the application.

Auxiliaries are substances which are suitable for imparting to the composition itself and/or to preparations derived therefrom (for example spray liquors, seed dressings) particular properties such as certain technical properties and/or also particular biological properties. Typical suitable auxiliaries are: extenders, solvents and carriers.

Suitable extenders are, for example, water, polar and nonpolar organic chemical liquids, for example from the classes of the aromatic and non-aromatic hydrocarbons (such as paraffins, alkylbenzenes, alkylnaphthalenes, chlorobenzenes), the alcohols and polyols (which, if appropriate, may also be substituted, etherified and/or esterified), the ketones (such as acetone, cyclohexanone), esters (including fats and oils) and (poly)ethers, the unsubstituted and substituted amines, amides, lactams (such as N-alkylpyrrolidones) and lactones, the sulfones and sulfoxides (such as dimethyl sulfoxide).

If the extender used is water, it is also possible to use, for example, organic solvents as auxiliary solvents. Essentially, suitable liquid solvents are: aromatics such as xylene, toluene or alkylnaphthalenes, chlorinated aromatics and chlorinated aliphatic hydrocarbons such as chlorobenzenes, chloroethylenes or methylene chloride, aliphatic hydrocarbons such as cyclohexane or paraffins, for example petroleum fractions, alcohols such as butanol or glycol and also their ethers and esters, ketones such as acetone, methyl ethyl ketone, methyl isobutyl ketone or cyclohexanone, strongly polar solvents such as dimethylformamide and dimethyl sulfoxide.

Suitable solid carriers are: for example, ammonium salts and ground natural minerals such as kaolins, clays, talc, chalk, quartz, attapulgite, montmorillonite or diatomaceous earth, and ground synthetic minerals, such as finely divided silica, alumina and silicates; suitable solid carriers for granules are: for example, crushed and fractionated natural rocks such as calcite, marble, pumice, sepiolite and dolomite, and also synthetic granules of inorganic and organic meals, and granules of organic material such as paper, sawdust, coconut shells, maize cobs and tobacco stalks; suitable emulsifiers and/or foam-formers are: for example, nonionic and anionic emulsifiers, such as polyoxyethylene fatty acid esters, polyoxyethylene fatty alcohol ethers, for example alkylaryl polyglycol ethers, alkylsulfonates, alkyl sulfates, arylsulfonates and also protein hydrolysates; suitable dispersants are nonionic and/or ionic substances, for example from the classes of the alcohol-POE and/or -POP ethers, acid and/or POP-POE esters, alkylaryl and/or POP-POE ethers, fat- and/or POP-POE adducts, POE- and/or POP-polyol derivatives, POE- and/or POP-sorbitan- or -sugar adducts, alkyl or aryl sulfates, alkyl- or arylsulfonates and alkyl or aryl phosphates or the corresponding PO-ether adducts. Furthermore, suitable oligo- or polymers, for example those derived from vinylic monomers, from acrylic acid, from EO and/or PO alone or in combination with, for example, (poly)alcohols or (poly)amines. It is also possible to employ lignin and its sulfonic acid derivatives, unmodified and modified celluloses, aromatic and/or aliphatic sulfonic acids and their adducts with formaldehyde.

Tackifiers such as carboxymethylcellulose and natural and synthetic polymers in the form of powders, granules or latices, such as gum arabic, polyvinyl alcohol and polyvinyl acetate, as well as natural phospholipids such as cephalins and lecithins, and synthetic phospholipids, can be used in the formulations.

Further additives may be mineral and vegetable oils. It is also possible to add colorants such as inorganic pigments, for example iron oxide, titanium oxide and Prussian Blue, and organic dyestuffs, such as alizarin dyestuffs, azo dyestuffs and metal phthalocyanine dyestuffs, and trace nutrients such as salts of iron, manganese, boron, copper, cobalt, molybdenum and zinc. Other possible additives are perfumes, mineral or vegetable, optionally modified oils and waxes.

The formulations may also comprise stabilizers, e.g. low-temperature stabilizers, preservatives, antioxidants, light stabilizers or other agents which improve chemical and/or physical stability.

The formulations generally comprise between 0.01 and 98% by weight of active compound, preferably between 0.1 and 95% and particularly preferably between 0.5 and 90%.

The active compounds according to the invention can also be used as a mixture with other known fungicides, for example, to improve the activity spectrum or to reduce or slow the development of resistance.

A mixture with other known active compounds such as nematicides, acaricides, herbicides, insecticides, bactericides or other fungicides, or with fertilizers and growth regulators, safeners or semiochemicals is also possible.

Exemplary application rates of the active compounds according to the invention are: when treating leaves: from 0.1 to 10 000 g/ha, preferably from 10 to 1000 g/ha, particularly preferably from 50 to 300 g/ha (when the application is carried out by watering or dripping, it is even possible to reduce the application rate, especially when inert substrates such as rock wool or perlite are used); when treating seed: from 2 to 200 g per 100 kg of seed, preferably from 2.5 to 150 g per 100 kg of seed, and particularly preferably from 2.5 to 25 g per 100 kg of seed, very particularly preferably from 2.5 to 12.5 g per 100 kg of seed; when treating the soil: from 0.1 to 10 000 g/ha, preferably from 1 to 5 000 g/ha.

The compositions according to the invention are suitable for protecting any plant variety which is employed in agriculture, in the greenhouse, in forests or in horticulture and, in particular, cereals (e.g. wheat, barley, rye, millet and oats), maize, cotton, soya beans, rice, potatoes, sunflowers, beans, coffee, beet (for example sugar beet and fodder beet), peanuts, vegetables (e.g. tomatoes, cucumbers, onions and lettuce), lawns, fruit and nut trees (e.g. apples pears peaches nectarines, apricots, hazelnut, pecan, macadamia, pistachio), soft fruit (e.g. strawberries, raspberries, blackcurrants, redcurrants), grapevines, bananas, cocoa and ornamental plants.

The active compounds of the invention, in combination with good plant tolerance and favourable toxicity to warm-blooded animals and being tolerated well by the environment, are suitable for protecting plants and plant organs, for increasing the harvest yields, for improving the quality of the harvested material and for controlling pests, in particular fungal diseases, which are encountered in agriculture, in horticulture, in animal husbandry, in forests, in gardens and leisure facilities, in the protection of stored products and of materials, and in the hygiene sector. They may be preferably employed as crop protection agents.

Use as Fungicides

The compounds of the invention have activity as fungicides.

The following are illustrative examples of agricultural pests that may be controlled by fungicidal compounds:

Oomycete diseases such as: Albugo diseases caused for example by Albugo Candida; Bremia diseases, caused for example by Bremia lactucae; Peronospora diseases, caused for example by Peronospora pisi or P. brassicae; Phytophthora diseases, caused for example by Phytophthora infestans; Plasmopara diseases, caused for example by Plasmopara viticola; Pseudoperonospora diseases, caused for example by Pseudoperonospora humuli or Pseudoperonospora cubensis; Pythium diseases, caused for example by Pythium ultimum; The compounds of the invention may be active against a broad spectrum of oomycete fungal diseases. Alternatively, they may be active specifically against certain oomycete diseases but not others.

Notable oomycete fungal diseases are:
Plasmopara viticola
Phytophthora infestans
Pythium ultimum
Bremia lactuca
Peronospora spp

In addition to their fungicidal activity, the compounds of the invention may also have some activity against other microbes, e.g. bacteria.

The fungicidal compounds of the invention may also be used in the treatment of fungal diseases of humans and animals (e.g. mammals). Likewise, the bactericidal compounds of the invention may be used in the treatment of bacterial diseases of humans and animals. Thus, the invention includes a method of treating a fungal or bacterial disease, the method comprising administering a therapeutic amount of an antifungal agent of the invention to a subject (e.g. a human subject) in need thereof. The compound may be formulated for topical administration to the infected area of the body or it may be formulated for oral or parenteral administration.

Synthesis

The skilled person will appreciate that adaptation of methods known in the art could be applied in the manufacture of the compounds of the present invention.

For example, the skilled person will be immediately familiar with standard textbooks such as “Comprehensive Organic Transformations—A Guide to Functional Group Transformations”, RC Larock, Wiley-VCH (1999 or later editions); “March's Advanced Organic Chemistry—Reactions, Mechanisms and Structure”, MB Smith, J. March, Wiley, (5th edition or later); “Advanced Organic Chemistry, Part B, Reactions and Synthesis”, F A Carey, R J Sundberg, Kluwer Academic/Plenum Publications, (2001 or later editions); “Organic Synthesis—The Disconnection Approach”, S Warren (Wiley), (1982 or later editions); “Designing Organic Syntheses” S Warren (Wiley) (1983 or later editions); “Heterocyclic Chemistry”, J. Joule (Wiley 2010 edition or later); (“Guidebook To Organic Synthesis” R K Mackie and D M Smith (Longman) (1982 or later editions), etc., and the references therein as a guide.

The skilled person is familiar with a range of strategies for synthesising organic and particularly heterocyclic molecules and these represent common general knowledge as set out in text books such as Warren “Organic Synthesis: The Disconnection Approach”; Mackie and Smith “Guidebook to Organic Chemistry”; and Clayden, Greeves, Warren and Wothers “Organic Chemistry”.

The skilled chemist will exercise his judgement and skill as to the most efficient sequence of reactions for synthesis of a given target compound and will employ protecting groups as necessary. This will depend inter alia on factors such as the nature of other functional groups present in a particular substrate. Clearly, the type of chemistry involved will influence the choice of reagent that is used in the said synthetic steps, the need, and type, of protecting groups that are employed, and the sequence for accomplishing the protection/deprotection steps. These and other reaction parameters will be evident to the skilled person by reference to standard textbooks and to the examples provided herein.

Sensitive functional groups may need to be protected and deprotected during synthesis of a compound of the invention. This may be achieved by conventional methods, for example as described in “Protective Groups in Organic Synthesis” by TW Greene and PGM Wuts, John Wiley & Sons Inc. (1999), and references therein.

Throughout this specification these abbreviations have the following meanings:

| | |
|---|---|
| CDI—carbonyldiimidazole | DCM—dichloromethane |
| DDQ—2,3-dichloro-5,6-dicyano-1,4-benzoquinone | DIPEA—diisopropylethylamine |
| DMAP—N,N-dimethyl-4-aminopyridine | DMF—N,N-dimethylformamide |
| DMSO—dimethylsulfoxide | Im—imidazole |
| LDA—Lithium diisopropylamide | mCPBA—m-chloroperbenzoic acid |
| NBS—N-bromosuccinimide | PE—petroleum ether |
| PMB—para-methoxybenzyl | RT—room temperature |
| TBAF—tetrabutylammonium fluoride | Tf—trifluoromethylsulfonyl |
| THF—tetrahydrofuran | TMS—trimethylsilyl |
| TCDI—thiocarbonyldiimidazole | TBSO—t-butyldimethylsilyloxy |
| HATU—1-[Bis(dimethylamino)methylene]- | min—minutes |
| 1H-1,2,3-triazolo[4,5-b]pyridinium 3-oxide | |
| hexafluorophosphate | |
| NaHMDS—sodium bis(trimethylsilyl)amide | LHMDS—lithium bis(trimethylsilyl)amide |

Certain compounds of the invention can be made according to the general synthetic scheme below. Certain compounds of the invention can be made according to or by methods analogous to the methods described in Examples 1-48.

General Synthetic Scheme

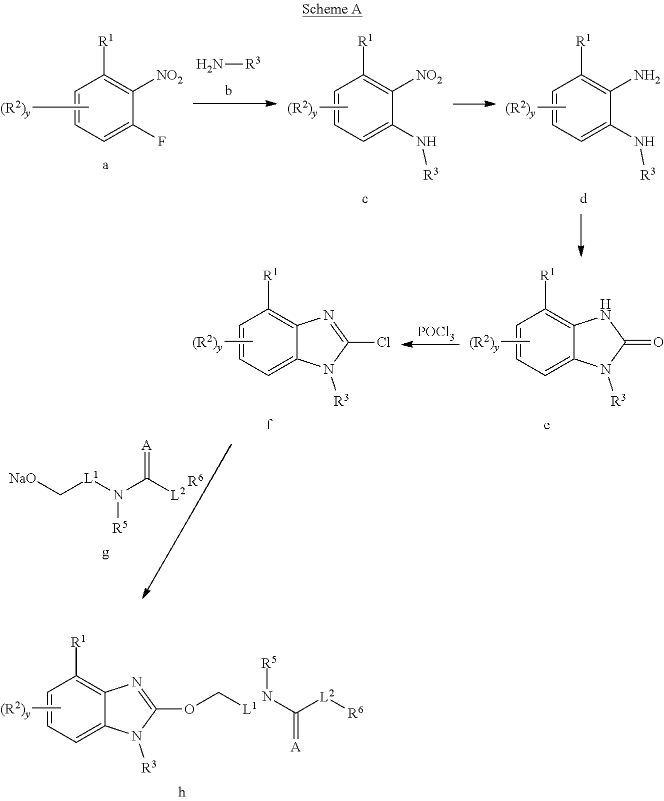

Certain compounds of the invention can be made starting from ortho-fluoro nitro benzenes a. Treatment with amine b in the presence of a base (e.g. NaH in DMF) can provide nitroanilines of formula c. Reduction of the nitro group to an amine (e.g. using ammonium formate and palladium on carbon in ethanol) can provide the diamines d. Compounds of formula e can be formed (e.g. by treating with carbonyl diimidazole in THF at room temperature). Treatment with phosphorous oxychloride (e.g. at reflux) can provide chlorobenzimidazoles of formula f. Reaction of compounds of formula f with sodium alkoxide g (in which LG is a leaving group, for example OTf, Cl, Br, I) (e.g. in DMF) provides compounds of formula h, a subset of compounds of the invention (Scheme A).

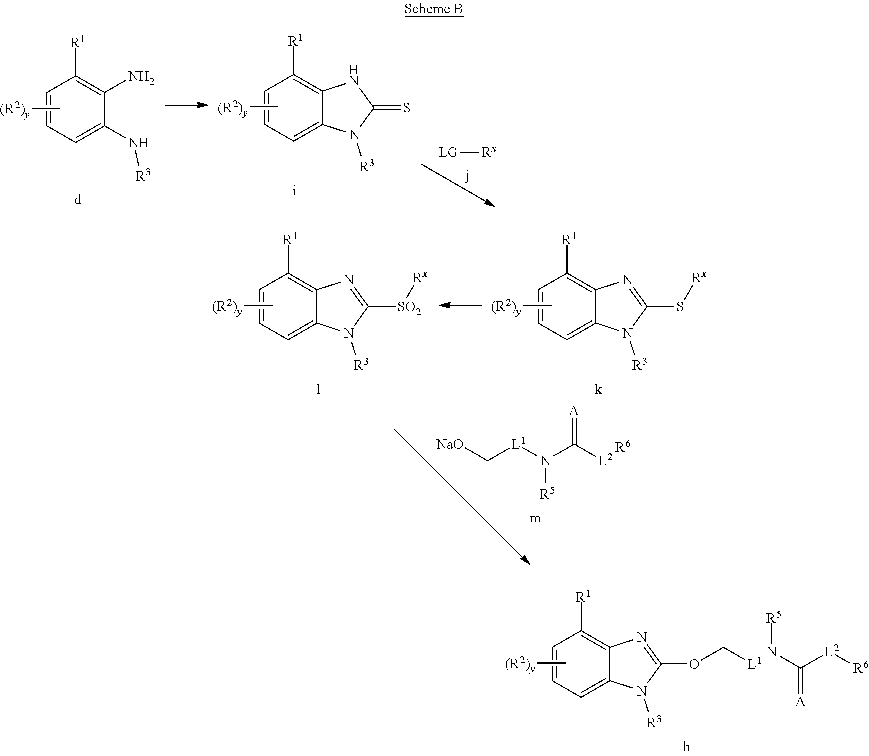

Certain compounds of the invention can be made starting from diamines d. Compounds of formula i can be formed (e.g. by treating with TCDI in THF). Reaction with electrophile j (in which LG is a leaving group, for example OTf, Cl, Br, I and R can be any convenient group) provides compounds of formula k. Treatment with mCPBA (e.g. in DCM at room temperature) can give sulfones of formula I. Reaction with a sodium alkoxide m (e.g. in DMF at room temperature) provides compounds of formula h, a subset of compounds of the invention (Scheme B).

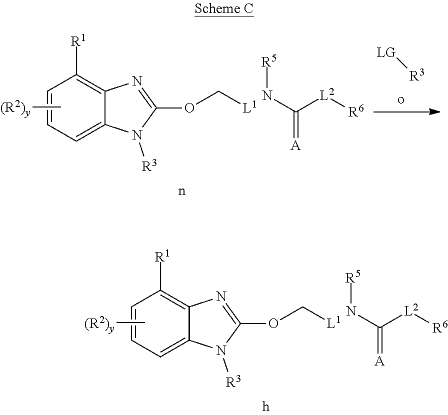

Alternatively, treatment of compounds of formula n with a heterocycle of formula o (in which LG is a leaving group such as F, Cl, Br, OTf, SO₂R^{x}) (e.g. in the presence of a base such as NaH in a suitable solvent such as DMF at a suitable temperature, e.g. 100° C.) provides compounds of formula h, a subset of compounds of the invention (Scheme C).

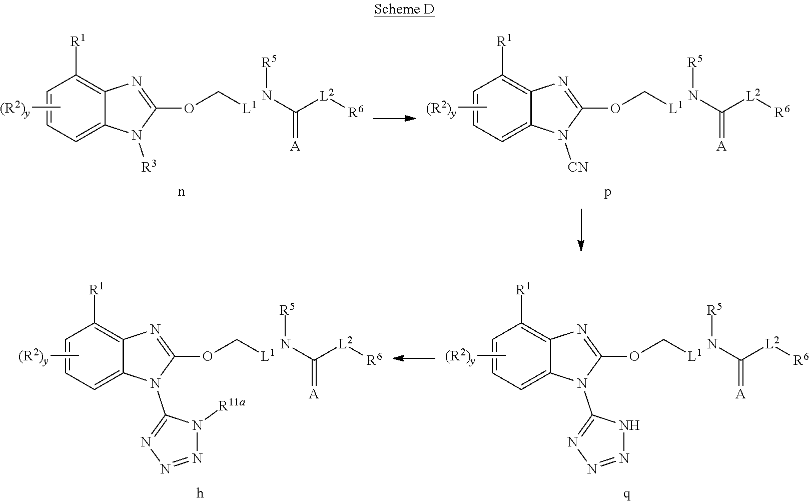

As a further alternative, treatment of compounds of formula n with cyanogen bromide (e.g. in acetone at 0° C.) can form compounds of formula p. Reaction with ammonium chloride followed by sodium azide (e.g. in DMF at room temperature) provides tetrazoles of formula q. Alkylation with R⁸-LG (where LG is a leaving group such as Cl, Br, I, OTf) (e.g. in the presence of a base such as K₂CO₃ in a solvent such as DMF at suitable temperature, e.g. 40° C.) provides compounds of formula h, a subset of compounds of the invention (Scheme D).

Analytical Procedures

Flash chromatography was carried out using a Biotage Isolera 4, with Biotage® SNAP KP-Sil cartridges, packed with 50 μm silica particles with a surface area of 500 m²/g, or alternative cartridges (e.g. Puriflash, produced by Interchim; Claricep, produced by Agela Technologies) where stated. Visualisation was carried out with UV light (254 nm) and by staining with either potassium permanganate, phosphomolybdic acid (PMA) or ninhydrin solutions.

All ¹H NMR spectra were obtained on a Bruker AVIII 400 with 5 mm QNP or Bruker AVI 500 with 5 mm QNP or Bruker DPX 300. Chemical shifts are expressed in parts per million (δ) and are referenced to the solvent. Coupling constants J are expressed in Hertz (Hz).

MS was carried out on a Waters Alliance ZQ MS (Methods A and B) or on a Waters Acquity UPLC-QDA UV-MS (Method D), using one of the methods below:

Method a (5 Minute Basic pH)

Column: YMC-Triart C18 50×2 mm, 5 μm. Flow rate: 0.8 mL/min. Injection volume: 5 μL.

| | | |
|---|---|---|
| Mobile Phase | A | H₂O |
| | B | CH₃CN |
| | C | 50% H₂O/50% CH₃CN + 1.0% ammonia (aq.) |

| Time (min) | A (%) | B (%) | C (%) |
|---|---|---|---|
| 0 | 95 | 0 | 5 |
| 4 | 0 | 95 | 5 |
| 4.4 | 0 | 95 | 5 |
| 4.5 | 95 | 5 | 0 |

| | |
|---|---|
| 4.5 | STOP |

Method B (5 Minute Acidic pH)

Column: YMC-Triart C18 50×2 mm, 5 μm. Flow rate: 0.8 mL/min. Injection volume: 5 μL.

| | | |
|---|---|---|
| Mobile Phase | A | H₂O |
| | B | CH₃CN |
| | C | 50% H₂O/50% CH₃CN + 1.0% formic acid |

| Time (min) | A (%) | B (%) | C (%) |
|---|---|---|---|
| 0 | 95 | 0 | 5 |
| 4 | 0 | 95 | 5 |
| 4.4 | 0 | 95 | 5 |
| 4.5 | 95 | 5 | 0 |

| | |
|---|---|
| 4.5 | STOP |

Method D (3.5 Minute Acidic pH)

Mobile phases: Water (A)/Acetonitrile (B) both with 0.1% (v/v) Formic Acid

| Time | % A | % B | Flow rate (mL/min) |
|---|---|---|---|
| Initial | 98 | 2 | 1.0 |
| 0.2 | 98 | 2 | 1.0 |
| 2.5 | 2 | 98 | 1.0 |
| 3.0 | 2 | 98 | 1.0 |
| 3.1 | 98 | 2 | 1.0 |
| 3.5 | 98 | 2 | 1.0 |

Column: SH C18 2.1×60 mm, 1.7 μm @50° C.

All reagents were obtained from commercial suppliers and used as supplied unless otherwise stated.

All examples are named using ChemBioDraw Ultra 14.0.

Reactions were conducted at ambient temperature (RT) unless otherwise stated.

### Synthetic Intermediates

### N-(3-Chloro-2-nitrophenyl)-1-methyl-1H-tetrazol-5-amine A

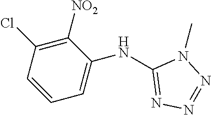 [SMILES: Cn1nnnc1Nc1cccc(Cl)c1[N+](=O)[O-]]

A stirred, ice-cooled suspension of 5-amino-1-methyltetrazole (0.847 g, 8.55 mmol) in dry DMF (15 mL) was treated with a 60% mineral oil dispersion of sodium hydride (0.786 g, 19.7 mmol) and stirred a further 5 min then 2-chloro-6-fluoronitrobenzene (1.50 g, 8.55 mmol) was added dropwise over ca. 2 min such that the internal temperature did not exceed 20° C. The cooling bath was then removed and the dark red solution was stirred whilst warming to RT over 45 min then diluted cautiously with water (80 mL) and washed with ether (80 mL). The aqueous (aq.) layer was acidified with 5M aq. HCl (4 mL, 20 mmol), extracted with EtOAc (2×80 mL) and the organics washed with water (2×80 mL) and brine then combined, dried (MgSO₄) and concentrated to give N-(3-chloro-2-nitrophenyl)-1-methyl-1H-tetrazol-5-amine A (2.11 g, 97%), as a yellow solid. No further purification was required.

¹H NMR (500 MHz, CDCl₃) β 8.37 (dd, J=8.6, 1.2 Hz, 1H), 8.23 (br s, 1H), 7.59-7.47 (m, 1H), 7.26 (dd, J=8.0, 1.2 Hz, 1H), 3.97 (s, 3H); LCMS (Method B): 2.45 min (255.1, MH⁺).

Following the same procedure as for Synthetic Intermediate A, with the appropriate starting material in place of 2-chloro-6-fluoronitrobenzene, there were thus obtained the following Intermediates (Int. A1-A5):

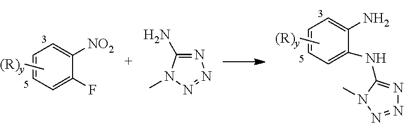

| Int. | (R)_{y} | Yield | RT^{a} | MH⁺ |
|---|---|---|---|---|
| A1 | 3-Methoxy-5-F | 97% | 2.62 (B) | 269.1 |
| A2 | 3-Allyloxy | 60%^{b} | 2.82 (B) | 277.1 |
| A4 | 3-Methyl | 79% | 2.53 (B) | 235.1 |
| A5 | 3-Bromo | 99% | 2.73 (B) | 301.0^{c} |

^{a}RT = LCMS retention time in minutes using indicated Method (A-D);
^{b}After chromatography;
^{c}One of a pair of Br isotope ions.

### 3-Chloro-N¹-(1-methyl-1H-tetrazol-5-yl)benzene-1,2-diamine B

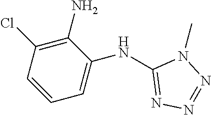

A solution of N-(3-chloro-2-nitrophenyl)-1-methyl-1H-tetrazol-5-amine A (2.00 g, 7.85 mmol) in methanol (150 mL) was treated with water (50 mL), iron powder (1.76 g, 31.5 mmol) and ammonium chloride (2.52 g, 47.1 mmol) then heated under reflux for 20 h. The mixture was cooled, filtered through Celite and concentrated. The solid was partitioned between EtOAc (100 mL; not fully soluble) and water (100 mL) and the organics washed further with water (100 mL) and brine then dried (MgSO₄) to give a maroon solid (0.98 g). The residual solid after decantation of the EtOAc solution was dissolved in ca. 20% MeOH/DCM, dried (MgSO₄) and concentrated to give an off-white solid (0.74 g). These two solids were combined to give 3-chloro-N¹-(1-methyl-1H-tetrazol-5-yl)benzene-1,2-diamine B (1.72 g, 97%).

¹H NMR (500 MHz, DMSO-d6) δ 8.40 (s, 1H), 7.19 (dd, J=7.9, 1.3 Hz, 1H), 7.12 (dd, J=8.0, 1.3 Hz, 1H), 6.58 (t, J=8.0 Hz, 1H), 5.28 (s, 2H), 3.87 (s, 3H); LCMS (Method B): 1.86 min (225.1, MH⁺).

In a subsequent experiment the extractive work-up was replaced with a simpler procedure: the partially concentrated filtrates, essentially free from MeOH, were filtered and the solid washed with water and dried to give B containing traces of iron salts (NMR signals broadened) but pure enough for the next step.

Following the same procedure as for Synthetic Intermediate B, with the appropriate starting material in place of N-(3-chloro-2-nitrophenyl)-1-methyl-1H-tetrazol-5-amine A, and with simplified work-up procedures where possible due to higher solubility of the product, there were thus obtained the following Intermediates (Int. B2 and B5):

| 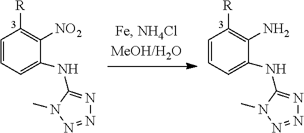 | | | | | |
|---|---|---|---|---|---|
| Int. | (R)_{y} | SM | Yield | RT^{a} | MH⁺ |
| B2 | 3-Allyloxy | A2 | 100%^{b} | 2.18 (B) | 247.1 |
| B5 | 3-Bromo | A5 | 59%^{c} | 2.21 (B) | 271.1^{d} |

^{a}RT = LCMS retention time in minutes using indicated Method (A-D);
^{b}Chromatography was not required;
^{c}No extractive work-up—product isolated by evaporating off the MeOH in vacuothen filtering and washing the solid with water;
^{d}One of a pair of Br isotope ions.

### 5-Fluoro-3-methoxy-N¹-(1-methyl-1H-tetrazol-5-yl)benzene-1,2-diamine B1

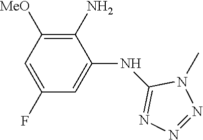 [SMILES: COc1cc(F)cc(Nc2nnnn2C)c1N]

A stirred suspension of N-(5-fluoro-3-methoxy-2-nitrophenyl)-1-methyl-1H-tetrazol-5-amine A1 (3.08 g, 11.5 mmol) in ethanol (150 mL) under nitrogen was treated with 10% palladium on activated charcoal (130 mg) then ammonium formate (2.9 g, 46 mmol) and the mixture was heated under reflux for 8 h. The mixture was cooled slightly and filtered through diatomaceous earth under nitrogen whilst still hot. The filter was washed with hot ethanol (2×15 mL) and the filtrate reduced in volume by evaporation until precipitation commenced. The mixture was then cooled in ice-water to 7° C. and the solid collected by filtration, washed with cold ethanol (2×10 mL) and dried in vacuo overnight to give 5-fluoro-3-methoxy-N¹-(1-methyl-1H-tetrazol-5-yl)benzene-1,2-diamine B1 (1.29 g, 47%) as an off-white solid.

¹H NMR (500 MHz, DMSO-d6) δ 6.91 (dd, J=10.6, 2.8 Hz, 1H), 6.66 (dd, J=10.5, 2.8 Hz, 1H), 4.50 (br s, 2H), 3.88 (s, 3H), 3.81 (s, 3H); LCMS (Method B): 1.72 min (239.1, MH⁺).

Following the same procedure as for Synthetic Intermediate B1, with the appropriate starting material in place of N-(5-fluoro-3-methoxy-2-nitrophenyl)-1-methyl-1H-tetrazol-5-amine A1, and with simplified work-up procedures where possible due to higher solubility of the product, there was thus obtained the following Intermediate (Int. B4):

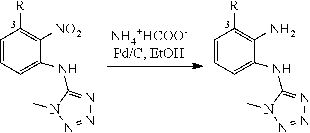

| Int. | (R)_{y} | SM | Yield | RT^{a} | MH⁺ |
|---|---|---|---|---|---|
| B4 | 3-Methyl | A5 | 79%^{b} | 2.57 (B) | 235.1 |

^{a}RT = LCMS retention time in minutes using indicated Method (A-D);
^{b}Chromatography was not required.

### 4-Chloro-1-(1-methyl-1H-tetrazol-5-yl)-1,3-dihydro-2H-benzo[d]imidazole-2-thione C

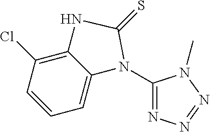 [SMILES: Cn1nnnc1-n1c(=S)[nH]c2c(Cl)cccc21]

A suspension of 3-chloro-N¹-(1-methyl-1H-tetrazol-5-yl)benzene-1,2-diamine B (0.50 g, 2.23 mmol) in dry THF (15 mL) was treated with thiocarbonyldiimidazole (0.6 g, 3.37 mmol) and stirred under nitrogen at RT then dry DMF (5 mL) was added. The mixture was heated to 70° C. under reflux for 68 h then cooled, diluted with water (120 mL) and extracted with EtOAc (2×100 mL). The organics were washed with water (100 mL) and brine then combined, dried (MgSO₄) and concentrated to give a dark red solid. This material was dry-loaded onto silica and chromatographed on silica (40 g Claricep cartridge) eluting with 1-2.5% MeOH/DCM to give 4-chloro-1-(1-methyl-1H-tetrazol-5-yl)-1,3-dihydro-2H-benzo[d]imidazole-2-thione C (342 mg, 58%) as a red solid.

¹H NMR (500 MHz, DMSO-d6) δ 14.20 (s, 1H), 7.41 (d, J=7.5 Hz, 1H), 7.24 (t, J=8.1 Hz, 1H), 7.07 (d, J=7.6 Hz, 1H), 4.09 (s, 3H); LCMS (Method B): 2.52 min (267.1, MH⁺).

Following the same procedure as for Synthetic Intermediate C, with the appropriate starting material in place of 3-chloro-N-(1-methyl-1H-tetrazol-5-yl)benzene-1,2-diamine B, and using DMF as solvent at 90° C. instead of THF/DMF at 70° C., there was thus obtained the following Intermediate (Int. C1):

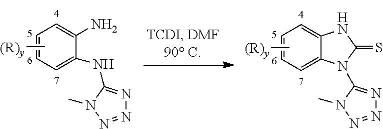

| Int. | (R)_{y} | SM | Yield | RT^{a} | MH⁺ |
|---|---|---|---|---|---|
| C1 | 4-Methoxy-6-F | B1 | 91%^{b} | 2.58 (B) | 281.1 |

^{a}RT = LCMS retention time in minutes using indicated Method (A-D);
^{b}Chromatography was not required.

### 4-Methyl-1-(1-methyl-1H-tetrazol-5-yl)-1,3-dihydro-2H-benzo[d]imidazole-2-thione C4

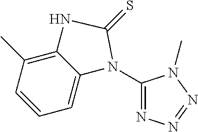 [SMILES: Cc1cccc2c1[nH]c(=S)n2-c1nnnn1C]

A suspension of 3-methyl-N¹-(1-methyl-1H-tetrazol-5-yl)benzene-1,2-diamine B4 (400 mg, 2.0 mmol) in ethanol (20 ml) and water (1 mL) was treated with ethylxanthic acid potassium salt (950 mg, 6.0 mmol) and stirred under reflux for 4 h. Saturated aq. NH₄Cl and water were added and the mixture was extracted with EtOAc (20 mL). The organic extracts were washed with brine, dried (MgSO₄) and concentrated in vacuo to give 4-methyl-1-(1-methyl-1H-tetrazol-5-yl)-1,3-dihydro-2H-benzo[d]imidazole-2-thione C4 (400 mg, 83%) as a dark yellow solid.

¹H NMR (500 MHz, CDCl₃) δ 10.15 (s, 1H), 7.19-7.12 (m, 2H), 6.89 (d, J=7.7 Hz, 1H), 4.17 (s, 3H), 2.47 (s, 3H); LCMS (Method B); 2.57 min (247.1, MH⁺).

### 4-Chloro-1-(1-methyl-1H-tetrazol-5-yl)-2-(methylthio)-1H-benzo[d]imidazole D

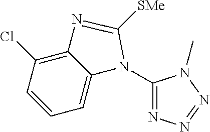 [SMILES: CSc1nc2c(Cl)cccc2n1-c1nnnn1C]

A stirred solution of 4-chloro-1-(1-methyl-1H-tetrazol-5-yl)-1,3-dihydro-2H-benzo[d]imidazole-2-thione C (347 mg, 1.30 mmol) in dry DMF (3 mL) was treated with caesium carbonate (593 mg, 1.8 mmol) and iodomethane (0.097 ml, 1.6 mmol). After 1 h the mixture was diluted with water (20 mL), stirred 5 min, and extracted with EtOAc (20 mL). The organics were washed with water (2×20 mL) and brine, dried (MgSO₄) and chromatographed on silica (20 g Claricep cartridge) eluting with 20-40% EtOAc/PE to give 4-chloro-1-(1-methyl-1H-tetrazol-5-yl)-2-(methylthio)-1H-benzo[d]imidazole D (183 mg, 50%) as a pale pink solid.

¹H NMR (500 MHz, CDCl₃) δ 7.37 (dd, J=7.9, 0.9 Hz, 1H), 7.20 (t, J=8.0 Hz, 1H), 6.95 (dd, J=8.1, 0.9 Hz, 1H), 3.99 (s, 3H), 2.87 (s, 3H); LCMS (Method B): 2.9 min (281.2, MH⁺).

Following the same procedure as for Synthetic Intermediate D, with the appropriate starting material in place of 4-chloro-1-(1-methyl-1H-tetrazol-5-yl)-1,3-dihydro-2H-benzo[d]imidazole-2-thione C, there were thus obtained the following Intermediates (Int. D1, D4):

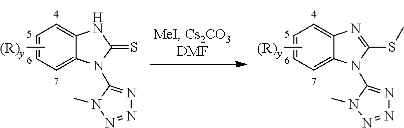

| Int. | (R)_{y} | SM | Yield | RT^{a} | MH⁺ |
|---|---|---|---|---|---|
| D1 | 4-Methoxy-6-F | C1 | 67% | 2.76 (B) | 295.1 |
| D4 | 4-Methyl | C4 | 56% | 3.19 (B) | 261.1 |

^{a}RT = LCMS retention time in minutes using indicated Method (A-D).

Alternatively, D was prepared in a one-pot process starting from B as follows:

A solution of 3-chloro-N¹-(1-methyl-1H-tetrazol-5-yl)benzene-1,2-diamine B (3.5 g, 15.6 mmol) in dry DMF (30 ml) was treated with thiocarbonyldiimidazole (4.2 g, 23.6 mmol) and stirred under nitrogen with heating to 90° C. for 3.5 h then cooled (ice-water), treated with iodomethane (1.55 mL, 24.9 mmol), stirred 1 min then treated with caesium carbonate (8.38 g, 25.7 mmol) and stirred at ambient temperature for 40 min. Water (30 mL) was added and stirring continued for 20 min. More water was added (200 mL) and the solid was collected and washed with water (2×100 mL) and dried (2 h in vacuo) to give 4-chloro-1-(1-methyl-1H-tetrazol-5-yl)-2-(methylthio)-1H-benzo[d]imidazole D (3.12 g, 71%) as a dark pink solid which did not require further purification.

Following the same Alternative Procedure as for Synthetic Intermediate D, with the appropriate starting material (SM) in place of 3-chloro-N¹-(1-methyl-1H-tetrazol-5-yl)benzene-1,2-diamine B, there were thus obtained the following Intermediates (Int. D2 and D5):

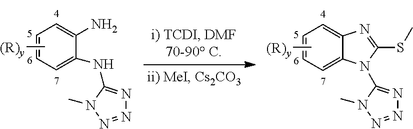

| Int. | (R)_{y} | SM | Yield | RT^{a} | MH⁺ |
|---|---|---|---|---|---|
| D2 | 4-Allyloxy | B2 | 82%^{b} | 3.10 (B) | 303.1 |
| D5 | 4-Bromo | B5 | 73% | 3.34 (B) | 327.0^{c} |

^{a}RT = LCMS retention time in minutes using indicated Method (A-D);
^{b}Extractive work-up followed by chromatography;
^{c}One of a pair of Br isotope ions.

### 4-Methoxy-1-(1-methyl-1H-tetrazol-5-yl)-2-(methylthio)-1H-benzo[d]imidazole D3

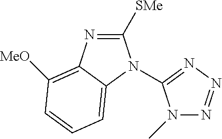 [SMILES: COc1cccc2c1nc(SC)n2-c1nnnn1C]

A solution of 1-(1-methyl-1H-tetrazol-5-yl)-2-(methylthio)-1H-benzo[d]imidazol-4-ol J (48 mg, 0.18 mmol) in dry DMF (1 mL) under nitrogen was treated with iodomethane (4 μL, 0.06 mmol) then caesium carbonate (89 mg, 0.28 mmol) and stirred at RT for 70 min then more iodomethane (4 μL, 0.06 mmol) was added. After a further 30 min water (25 mL) was added and the mixture was stirred for 10 min then extracted with EtOAc (2×30 mL). The organics were washed with water (3×25 mL) and brine, dried (MgSO₄) and concentrated to give 4-methoxy-1-(1-methyl-1H-tetrazol-5-yl)-2-(methylthio)-1H-benzo[d]imidazole D3 (46 mg, 91%) as an orange-yellow solid.

¹H NMR (500 MHz, CDCl₃) β 7.20 (t, J=8.1 Hz, 1H), 6.81 (d, J=8.0 Hz, 1H), 6.63 (dd, J=8.1, 0.6 Hz, 1H), 4.06 (s, 3H), 3.96 (s, 3H), 2.83 (s, 3H); LCMS (Method B): 2.66 min (277.1, MH⁺).

### 4-Chloro-1-(1-methyl-1H-tetrazol-5-yl)-2-(methylsulfonyl)-1H-benzo[d]imidazole E

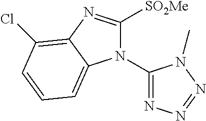 [SMILES: Cn1nnnc1-n1c(S(C)(=O)=O)nc2c(Cl)cccc21]

A stirred solution of 4-chloro-1-(1-methyl-1H-tetrazol-5-yl)-2-(methylthio)-1H-benzo[d]imidazole D (244 mg, 0.87 mmol) in DCM (5 mL) was treated with 75% strength 3-chloroperbenzoic acid (500 mg, 2.2 mmol) at RT for 24 h then diluted with DCM (20 mL), washed with saturated aq. sodium hydrogen carbonate solution (20 mL) and brine, dried (MgSO₄) and chromatographed on silica (20 g Claricep cartridge) eluting with 20-50% EtOAc/PE to give 4-chloro-1-(1-methyl-1H-tetrazol-5-yl)-2-(methylsulfonyl)-1H-benzo[d]imidazole E (235 mg, 86%) as a white foam.

¹H NMR (500 MHz, CDCl₃) δ 7.59 (dd, J=7.8, 0.9 Hz, 1H), 7.50 (t, J=8.1 Hz, 1H), 7.07 (dd, J=8.3, 0.9 Hz, 1H), 4.01 (s, 3H), 3.48 (s, 3H); LCMS (Method B): 2.71 min (313.1, MH⁺).

Following the same procedure as for Synthetic Intermediate E, with the appropriate starting material in place of 4-chloro-1-(1-methyl-1H-tetrazol-5-yl)-2-(methylthio)-1H-benzo[d]imidazole D, there were thus obtained the following Intermediates (Int. E1-E5):

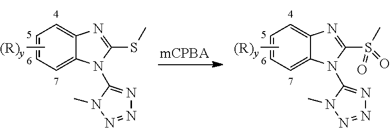

| Int. | (R)_{y} | SM | Yield | RT^{a} | MH⁺ |
|---|---|---|---|---|---|
| E1 | 4-MeO-6-F | D1 | 75% | 2.63 (B) | 327.1 |
| E2 | 4-Allyloxy | D2 | 46% | 2.96 (B) | 335.1 |
| E3 | 4-MeO | D3 | 74% | 2.55 (B) | 309.0 |
| E4 | 4-Me | D4 | 62% | 2.92 (B) | 293.1 |
| E5 | 4-Bromo | D5 | 89% | 3.12 (B) | 359.0^{b} |

^{a}RT = LCMS retention time in minutes using indicated Method (A-D);
^{b}One of a pair of Br isotope ions.

### N-(6-(((tert-Butyldimethylsilyl)oxy)methyl)pyridin-2-yl)cyclopropanecarboxamide F

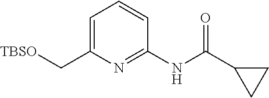 [SMILES: CC(C)(C)[Si](C)(C)OCc1cccc(NC(=O)C2CC2)n1]

A solution of 6-(((tert-butyldimethylsilyl)oxy)methyl)pyridin-2-amine (3.46 g, 14.5 mmol) in DCM (30 mL) was treated with cyclopropanecarbonyl chloride (1.32 mL, 14.5 mmol) and triethylamine (2.63 mL, 18.9 mmol) and stirred at RT for 1 hour. More DCM (40 mL) was added and the solution was washed with saturated aq. sodium hydrogen carbonate solution (50 mL), dried (MgSO₄) and concentrated in vacuo to give N-(6-(((tert-butyldimethylsilyl)oxy)methyl)pyridin-2-yl)cyclopropanecarboxamide F (4.02 g, 90%) as a white solid.

¹H NMR (500 MHz, CDCl₃) β 8.03 (d, J=8.3 Hz, 1H), 7.70 (t, J=7.9 Hz, 1H), 7.21 (d, J=7.5 Hz, 1H), 4.71 (s, 2H), 1.57-1.47 (m, 1H), 1.10 (m, 2H), 0.95 (s, 9H), 0.89 (dt, J=7.2, 4.2 Hz, 2H), 0.12 (s, 6H); LCMS (Method A): 3.90 min, (307.4, MH⁺).

### N-(6-(Hydroxymethyl)pyridin-2-yl)cyclopropanecarboxamide G

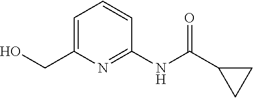 [SMILES: O=C(Nc1cccc(CO)n1)C1CC1]

TBAF [1M solution in THF] (15.74 mL, 15.74 mmol) was added to a solution of N-(6-(((tert-butyldimethylsilyl)oxy)methyl)pyridin-2-yl)cyclopropanecarboxamide F (4.02 g, 13.12 mmol) in THF (40 mL) at 0° C. and the reaction mixture was stirred at 0° C. for 3 hours. The mixture was diluted with ethyl acetate (100 mL), washed with water (40 mL) and brine (40 mL), dried (MgSO₄) and concentrated in vacuo to give a pale yellow oil which was chromatographed on silica eluting with 25-100% EtOAc/PE to give N-(6-(hydroxymethyl)pyridin-2-yl)cyclopropanecarboxamide G (1.95 g, 77%) as a pale yellow oil.

¹H NMR (500 MHz, CDCl₃) δ 8.45 (s, 1H), 8.07 (d, J=8.2 Hz, 1H), 7.67 (td, J=8.1, 1.6 Hz, 1H), 6.96 (d, J=7.5 Hz, 1H), 4.69 (s, 2H), 3.64 (s, 1H), 1.64-1.54 (m, 1H), 1.13-1.07 (m, 2H), 0.93-0.84 (m, 2H); LCMS (Method A): 1.03 min (193.2, MH⁺).

### N-(6-(Hydroxymethyl)pyridin-2-yl)pentanamide G1

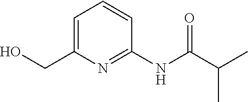

HATU (2.30 g, 6.05 mmol) was added to a stirred solution of (6-aminopyridin-2-yl)methanol (373 mg, 3.00 mmol), 4-methylvaleric acid (0.794 mL, 6.31 mmol) and N,N-diisopropylethylamine (3.14 mL, 18.0 mmol) in dry DMF (5 mL). The reaction mixture was stirred at RT for 22 h then diluted with EtOAc (30 mL) and washed with water (4×30 mL) and brine. The aq. layers were back-extracted with EtOAc (30 mL). Organics were dried (MgSO₄) and concentrated to give crude (6-(4-methylpentanamido)pyridin-2-yl)methyl 4-methylpentanoate (1 g, 3.12 mmol, 104% yield) as an orange oil containing some solid material, used directly in the next step. LCMS (Method B): 4.04 min (321.3, MH⁺ for amido-ester intermediate). A solution of crude (6-(4-methylpentanamido)pyridin-2-yl)methyl 4-methylpentanoate (0.961 g, 3.0 mmol) in THF (10 mL), methanol (5 mL) and water (5 mL) was treated with lithium hydroxide monohydrate (0.34 g, 8.1 mmol) and stirred at RT for 90 min then concentrated in vacuo to <10 mL. Water (30 mL) was added and the basic solution was extracted with EtOAc (2×30 mL). The organics were washed with water (30 mL) and brine, dried (MgSO₄) and concentrated to give crude product as an orange oil (642 mg). Chromatography on silica (20 g Claricep cartridge) eluting with 30-70% EtOAc/PE gave N-(6-(hydroxymethyl)pyridin-2-yl)-4-methylpentanamide G1 (0.487 g, 73%), as a near-colourless viscous oil.

¹H NMR (500 MHz, CDCl₃) δ 8.12 (d, J=8.2 Hz, 1H), 8.02 (s, 1H), 7.69 (t, J=7.9 Hz, 1H), 6.97 (dd, J=7.5, 0.7 Hz, 1H), 4.68 (s, 2H), 3.41 (s, 1H), 2.41 (dd, J=9.8, 5.6 Hz, 2H), 1.68-1.61 (m, 3H), 0.94 (d, J=6.5 Hz, 6H); LCMS (Method B): 2.54 min (223.2, MH⁺).

### N-(6-(Hydroxymethyl)pyridin-2-yl)cyclopentanecarboxamide G2

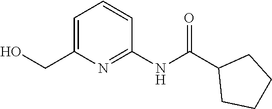 [SMILES: O=C(Nc1cccc(CO)n1)C1CCCC1]

A partial solution of (6-aminopyridin-2-yl)methanol (426 mg, 3.43 mmol) and N,N-diisopropylethylamine (1.44 mL, 8.2 mmol) in DCM (25 mL) was treated dropwise over 3 min with cyclopentane carbonyl chloride (0.90 mL, 7.9 mmol) and stirred at RT for 18 h to give a straw-coloured solution. 4-(Dimethylamino)pyridine (42 mg, 0.34 mmol) was added and stirring was continued a further 3 h then the mixture was concentrated in vacuo, treated with THF (10 ml), methanol (5 mL), water (5 mL) and lithium hydroxide monohydrate (720 mg, 17 mmol) and stirred at RT for 18 h. The mixture was concentrated in vacuo to about 5 mL, diluted with water (30 mL) and extracted with EtOAc (2×30 mL). The organics were washed with water (30 mL) and brine then combined, dried (MgSO₄) and chromatographed on silica (20 g Claricep cartridge) eluting with 30-70% EtOAc/PE to give N-(6-(hydroxymethyl)pyridin-2-yl)cyclopentanecarboxamide G2 (600 mg, 79%), as a near-colourless solid.

¹H NMR (500 MHz, CDCl₃) δ 8.21 (s, 1H), 8.11 (d, J=8.2 Hz, 1H), 7.66 (t, J=7.9 Hz, 1H), 6.96 (d, J=7.5 Hz, 1H), 4.67 (s, 2H), 3.82 (s, 1H), 2.73 (p, J=8.1 Hz, 1H), 1.99-1.84 (m, 4H), 1.83-1.71 (m, 2H), 1.69-1.52 (m, 2H); LCMS (Method B): 2.24 min (221.1, MH⁺).

The following intermediates (Int. G3-G12) were prepared in a similar fashion to G1 or G2. In many cases the procedures were suboptimal in that less than 2 eq. of RCOX was used, i.e. before it was appreciated that O-acylation occurred more rapidly than N-acylation. Hence a preferred method involves ensuring full conversion to the amido-ester di-acylated intermediate such that no amino-ester remains (which would otherwise subsequently be hydrolysed back to amino-alcohol).

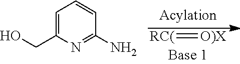

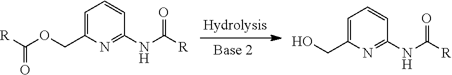

| Int. | RC(═O)X | Method | Yield | RT^{a} | Structure |
|---|---|---|---|---|---|
| G3 | 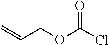 | G2^{b,c,d} | 51%^{e} | 2.38 (A) | 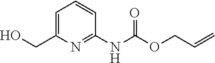 |
| G4 | 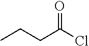 | G2^{c,f} | 25%^{e} | 2.15 (A) | 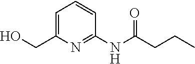 |
| G5 | 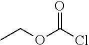 | G2^{f,g} | 49%^{e} | 2.31 (A) | 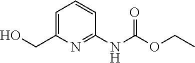 |
| G6 | 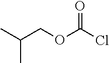 | G2^{f,g} | 61%^{e} | 2.94 (A) | 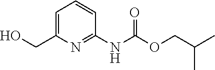 [SMILES: CC(C)COC(=O)Nc1cccc(CO)n1] |
| G7 | 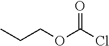 | G2^{f,g} | 47%^{e} | 2.64 (A) | 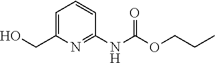 |
| G8 | 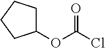 | G2^{f,g} | 17%^{e} | 2.87 (A) | 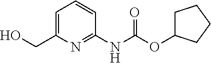 [SMILES: O=C(Nc1cccc(CO)n1)OC1CCCC1] |
| G9 | 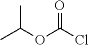 | G2^{f,h} | 7% | 2.59 (A) | 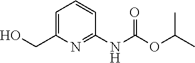 |
| G10 | 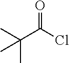 | G2^{f,h} | 35% | 2.27 (A) | 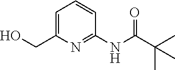 |
| G11 | 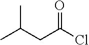 | G2ⁱ | 94% | 1.88 (B) | 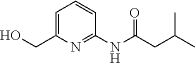 |
| G12 | 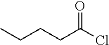 | G2ⁱ | 87% | 2.08 (B) | 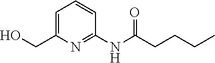 |

^{a}RT = LCMS retention time in minutes using indicated Method (A-D);
^{b}Solvent for Acylation was THF;
^{c}Pyridine was used as Base 1, using 1 molar equivalent of RC(═O)X, purifying the intermediate by chromatography on silica;
^{d}Base 2 was sodium hydroxide, heated to 60° C. in aq. THF;
^{e}Based on RC(═O)X;
^{f}Hydrolysis was carried out in 4:1:1 THF:MeOH:water at 50° C.;
^{g}Pyridine was used as Base 1, using 1 molar equivalent of RC(═O)X;
^{h}Pyridine was used as Base 1;
ⁱDMAP was not added.

### N-(4-(Hydroxymethyl)thiazol-2-yl)pentanamide G13

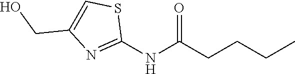

Following the same procedure as for Synthetic Intermediate G2, with the appropriate starting materials in place of (6-aminopyridin-2-yl) methanol and cyclopentane carbonyl chloride and without addition of DMAP, there was thus obtained N-(4-(hydroxymethyl)thiazol-2-yl)pentanamide G13 (614 mg, 73% yield) as an off-white solid.

¹HNMR (500 MHz, CDCl₃) δ 9.35 (br s, 1H), 6.80 (s, 1H), 4.65 (s, 2H), 2.46 (t, J=7.5 Hz, 2H), 1.72 (dt, J=15.1, 7.6 Hz, 2H), 1.40 (dd, J=15.0, 7.5 Hz, 2H), 0.94 (t, J=7.4 Hz, 3H); LCMS (Method B): 2.28 min (215.1, MH⁺).

### N-(4-(hydroxymethyl)thiazol-2-yl)-4-methylpentanamide G14

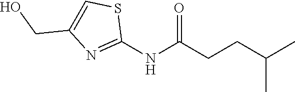

Following the same procedure as for Synthetic Intermediate G1, using (2-aminothiazol-4-yl)methanol as starting material in place of (6-aminopyridin-2-yl)methanol, there was thus obtained N-(4-(hydroxymethyl)thiazol-2-yl)-4-methylpentanamide G14 (494 mg, 64%) as a pale yellow solid.

¹H NMR (500 MHz, CDCl₃) β 6.80 (s, 1H), 4.65 (s, 2H), 2.47-2.43 (m, 2H), 1.65-1.59 (m, 3H), 0.92 (d, J=6.2 Hz, 6H); LCMS (Method B): 2.53 min (229.1, MH⁺).

### 6-(((4-Chloro-1-(1-methyl-1H-tetrazol-5-yl)-1H-benzo[d]imidazol-2-yl)oxy)methyl)pyridin-2-amine H

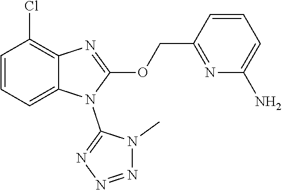 [SMILES: Cn1nnnc1-n1c(OCc2cccc(N)n2)nc2c(Cl)cccc21]

An ice-cooled solution of (6-aminopyridin-2-yl)methanol (164 mg, 1.32 mmol) and 4-chloro-1-(1-methyl-1H-tetrazol-5-yl)-2-(methylsulfonyl)-1H-benzo[d]imidazole (413 mg, 1.32 mmol) in dry DMF (3 mL) was treated with a 60% mineral oil dispersion of sodium hydride (61 mg, 1.52 mmol) and stirred for 90 min then saturated aqueous ammonium chloride solution (2 mL) was added and the mixture was diluted with EtOAc (50 mL), washed with water (4×50 mL) and brine. The aq. layers were back-extracted with EtOAc (40 mL). Organics were combined and dried (MgSO₄) to give 6-(((4-chloro-1-(1-methyl-1H-tetrazol-5-yl)-1H-benzo[d]imidazol-2-yl)oxy)methyl)pyridin-2-amine (455 mg, 95%), purity ca. 90%; further purification unnecessary.

¹H NMR (500 MHz, CDCl₃) δ 7.58-7.52 (m, 1H), 7.36 (dd, J=6.8, 2.2 Hz, 1H), 7.24-7.18 (m, 2H), 6.81 (d, J=7.2 Hz, 1H), 6.61 (d, J=8.4 Hz, 1H), 5.66 (s, 2H), 5.12 (br s, 2H), 4.14 (s, 3H); LCMS (Method B): 3.08 min (357.1, MH⁺).

### 1-(Allyloxy)-3-fluoro-2-nitrobenzene I

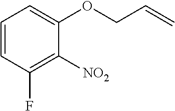

Potassium carbonate (2.02 g, 14.6 mmol) was added to a stirred solution of 3-fluoro-2-nitrophenol (2.09 g, 13.3 mmol) and allyl bromide (1.17 mL, 13.6 mmol) in dry DMF (8 mL) and the mixture was warmed to 50° C. for 3.5 h. More allyl bromide (0.35 mL, 4.0 mmol) was added and heating resumed for a further 60 min then the mixture was cooled, diluted with water (70 mL) and stirred for 10 min EtOAc (80 mL) was added and the mixture was separated and the organics washed further with water (3×70 mL) and brine, dried (MgSO₄) and concentrated to give 1-(allyloxy)-3-fluoro-2-nitrobenzene (2.74 g, 99%) as a pale amber oil.

¹H NMR (500 MHz, CDCl₃) β 7.37 (td, J=8.6, 6.2 Hz, 1H), 6.87-6.77 (m, 2H), 5.98 (ddt, J=17.3, 10.4, 5.1 Hz, 1H), 5.41 (ddd, J=17.3, 2.9, 1.7 Hz, 1H), 5.32 (ddd, J=10.6, 2.7, 1.4 Hz, 1H), 4.65 (dt, J=5.1, 1.6 Hz, 2H); LCMS (Method B): 3.48 min; no mol. ion detected for expected mass.

### 1-(1-Methyl-1H-tetrazol-5-yl)-2-(methylthio)-1H-benzo[d]imidazol-4-ol J

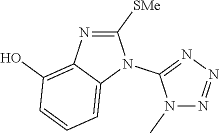 [SMILES: CSc1nc2c(O)cccc2n1-c1nnnn1C]

A solution of 4-(allyloxy)-1-(1-methyl-1H-tetrazol-5-yl)-2-(methylthio)-1H-benzo[d]imidazole D2 (355 mg, 1.17 mmol) in dry DMF (3 mL) was deoxygenated (3 cycles of vacuum/nitrogen) and treated with 2,2-dimethyl-1,3-dioxane-4,6-dione (338 mg, 2.35 mmol) then tetrakis(triphenylphosphine)palladium (136 mg, 0.12 mmol) and deoxygenated again then stirred at RT for 2 h. The mixture was diluted with EtOAc (30 mL), washed with water (4×30 mL) and brine, dried (MgSO₄) and chromatographed on silica (20 g Claricep cartridge) eluting with 10-50% EtOAc/DCM to give 1-(1-methyl-1H-tetrazol-5-yl)-2-(methylthio)-1H-benzo[d]imidazol-4-ol J (184 mg, 60%) as a pale yellow solid.

¹H NMR (500 MHz, DMSO-d6) δ 10.16 (s, 1H), 7.07 (t, J=8.0 Hz, 1H), 6.76 (dd, J=8.1, 0.8 Hz, 1H), 6.74 (dd, J=8.0, 0.8 Hz, 1H), 3.98 (s, 3H), 2.74 (s, 3H); LCMS (Method B): 2.33 min (266.1, MH⁺).

### Exemplary Compounds

### Example 1—N-(6-(((4-Chloro-1-(1-methyl-1H-tetrazol-5-yl)-1H-benzo[d]imidazol-2-yl)oxy)methyl)pyridin-2-yl)cyclopropanecarboxamide 1

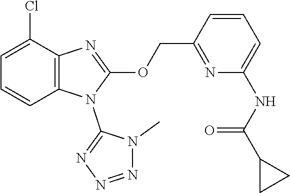 [SMILES: Cn1nnnc1-n1c(OCc2cccc(NC(=O)C3CC3)n2)nc2c(Cl)cccc21]

An ice-cooled solution of N-(6-(hydroxymethyl)pyridin-2-yl)cyclopropanecarboxamide G (37 mg, 0.19 mmol) and 4-chloro-1-(1-methyl-1H-tetrazol-5-yl)-2-(methylsulfonyl)-1H-benzo[d]imidazole E (60 mg, 0.19 mmol) in dry DMF (1 mL) was treated with a 60% mineral oil dispersion of sodium hydride (11 mg, 0.28 mmol) and stirred at 0° C. for 5 min then warmed to RT for 1 h. Saturated aq. ammonium chloride solution was added (2 mL) and the mixture was diluted with EtOAc (20 mL), washed with water (3×20 mL) and brine, dried (MgSO₄), chromatographed on silica (12 g Claricep cartridge) eluting with 0-2% DCM/MeOH and triturated with DCM to give N-(6-(((4-chloro-1-(1-methyl-1H-tetrazol-5-yl)-1H-benzo[d]imidazol-2-yl)oxy)methyl)pyridin-2-yl)cyclopropanecarboxamide 1 (50 mg, 61%) as a white solid.

¹H NMR (500 MHz, DMSO-d6) δ 10.86 (s, 1H), 8.06 (d, J=8.3 Hz, 1H), 7.86-7.77 (m, 1H), 7.40 (dd, J=8.0, 0.9 Hz, 1H), 7.36 (dd, J=8.1, 0.9 Hz, 1H), 7.25 (t, J=8.0 Hz, 1H), 7.22 (d, J=7.3 Hz, 1H), 5.67 (s, 2H), 4.08 (s, 3H), 2.04-1.96 (m, 1H), 0.84-0.78 (m, 4H); LCMS (Method D): 1.86 min (425.2, MH⁺).

Following the general procedure of Example 1, using the appropriate starting materials (X is a leaving group such as halo, methylsulfonyl, arylsulfonyl), and with chromatography in a suitable solvent system (more typically EtOAc/DCM or EtOAc/PE) followed by trituration (e.g. with ether or DCM/PE) if required, there were thus obtained the following Examples (Ex. 2-21):

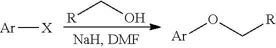

| Ex. | Ar—X | 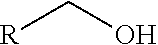 | Yield | ¹H NMR δ (CDCl₃) | RT^{a} | 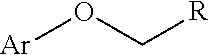 |
|---|---|---|---|---|---|---|
| 2 | E | [148167-30-0]^{b} | 73% | δ 8.86 (s, 1H), 8.28 (d, J = 8.1 Hz, 1H), 7.79 (t, J = 7.9 Hz, 1H), 7.41-7.32 (m, 3H), 7.23- 7.16 (m, 3H), 7.07 (dd, J = 7.8, 7.0 Hz, 1H), 7.04-6.99 (m, 2H), 5.70 (s, 2H), 4.65 (s, 2H), 3.99 (s, 3H) | 2.17 (B) | 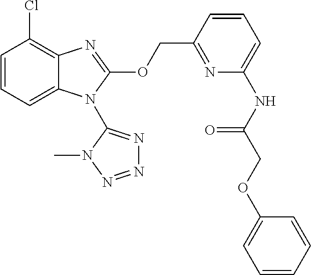 [SMILES: Cn1nnnc1-n1c(OCc2cccc(NC(=O)COc3ccccc3)n2)nc2c(Cl)cccc21] |
| 3 | E | [203321-83-9]^{b} | 66% | δ 7.95 (d, J = 8.4 Hz, 1H), 7.70 (t, J = 7.9 Hz, 1H), 7.34 (dd, J = 7.6, 1.4 Hz, 1H), 7.25-7.14 (m, 3H), 7.07 (d, J = 7.3 Hz, 1H), 5.65 (s, 2H), 4.04 (s, 3H), 1.53 (s, 9H) | 3.96 (B) | 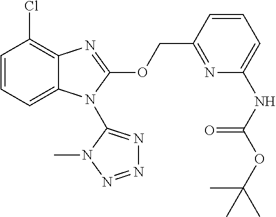 [SMILES: Cn1nnnc1-n1c(OCc2cccc(NC(=O)OC(C)(C)C)n2)nc2c(Cl)cccc21] |
| 4 | E1 | [203321-83-9]^{b} | 62% | δ 7.92 (d, J = 8.4 Hz, 1H), 7.70-7.64 (m, 1H), 7.25 (s, 1H), 7.00 (d, J = 7.3 Hz, 1H), 6.68 (dd, J = 7.9, 2.2 Hz, 1H), 6.59 (dd, J = 11.5, 2.2 Hz, 1H), 5.58 (s, 2H), 4.03 (s, 3H), 4.01 (s, 3H), 1.51 (s, 9H) | 3.57 (B) | 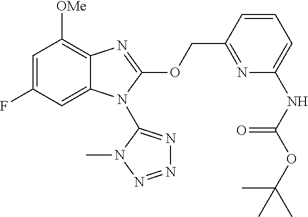 [SMILES: COc1cc(F)cc2c1nc(OCc1cccc(NC(=O)OC(C)(C)C)n1)n2-c1nnnn1C] |
| 5 | E1 | [148167-30-0]^{b} | 57% | δ 8.90 (s, 1H), 8.28 (d, J = 8.3 Hz, 1H), 7.77 (t, J = 7.9 Hz, 1H), 7.40-7.32 (m, 2H), 7.15 (d, J = 7.4 Hz, 1H), 7.07 (t, J = 7.4 Hz, 1H), 7.02 (dd, J = 8.7, 0.9 Hz, 2H), 6.67 (dd, J = 7.9, 2.2 Hz, 1H), 6.60 (dd, J = 11.5, 2.2 Hz, 1H), 5.65 (s, 2H), 4.65 (s, 2H), 4.02 (s, 3H), 4.00 (s, 3H) | 3.45 (B) | 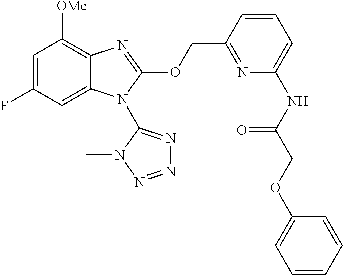 [SMILES: COc1cc(F)cc2c1nc(OCc1cccc(NC(=O)COc3ccccc3)n1)n2-c1nnnn1C] |
| 6 | E1 | G | 62% | δ 8.28 (s, 1H), 8.12 (d, J = 8.3 Hz, 1H), 7.67 (t, J = 7.9 Hz, 1H), 7.01 (d, J = 7.4 Hz, 1H), 6.67 (dd, J = 7.9, 2.2 Hz, 1H), 6.60 (dd, J = 11.5, 2.2 Hz, 1H), 5.64 (s, 2H), 4.05 (s, 3H), 4.00 (s, 3H), 1.67-1.60 (m, 1H), 1.12-1.06 (m, 2H), 0.94-0.89 (m, 2H) | 3.17 (B) | 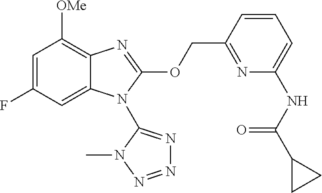 [SMILES: COc1cc(F)cc2c1nc(OCc1cccc(NC(=O)C3CC3)n1)n2-c1nnnn1C] |
| 7 | E2 | [203321-83-9]^{b} | 57% | δ 7.91 (d, J = 8.4 Hz, 1H), 7.71-7.61 (m, 1H), 7.21 (br s, 1H), 7.15 (t, J = 8.2 Hz, 1H), 7.02 (d, J = 6.9 Hz, 1H), 6.90 (dd, J = 8.1, 0.8 Hz, 1H), 6.83 (dd, J = 8.3, 0.6 Hz, 1H), 6.14 (ddt, J = 17.2, 10.6, 5.4 Hz, 1H), 5.62 (s, 2H), 5.45 (dq, J = 17.3, 1.6 Hz, 1H), 5.31 (dq, J = 10.5, 1.3 Hz, 1H), 4.85 (dt, J = 5.4, 1.5 Hz, 2H), 4.01 (s, 3H), 1.52 (s, 9H) | 3.74 (B) | 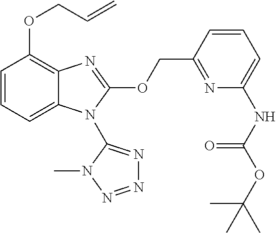 [SMILES: C=CCOc1cccc2c1nc(OCc1cccc(NC(=O)OC(C)(C)C)n1)n2-c1nnnn1C] |
| 8 | E3 | [203321-83-9]^{b} | 63% | δ 7.91 (d, J = 8.4 Hz, 1H), 7.70-7.63 (m, 1H), 7.23 (br s, 1H), 7.19 (t, J = 8.2 Hz, 1H), 7.01 (d, J = 7.2 Hz, 1H), 6.91 (dd, J = 8.1, 0.7 Hz, 1H), 6.85- 6.79 (m, 1H), 5.62 (s, 2H), 4.03 (s, 3H), 4.01 (s, 3H), 1.51 (s, 9H) | 3.37 (B) | 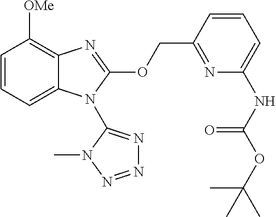 [SMILES: COc1cccc2c1nc(OCc1cccc(NC(=O)OC(C)(C)C)n1)n2-c1nnnn1C] |
| 9 | E | G1 | 79% | δ 8.16 (d, J =8 .2 Hz, 1H), 8.09 (br s, 1H), 7.69 (t, J = 7 .8 Hz, 1H), 7.37-7.29 (m, 1H), 7.22- 7.14 (m, 2H), 7.07 (d, J = 7.4 Hz, 1H), 5.67 (s, 2H), 4.03 (s, 3H), 2.47-2.34 (m, 2H), 1.65- 1.57 (m, 3H), 0.92 (d, J = 3.9 Hz, 6H) | 3.91 (B) | 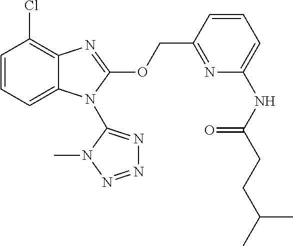 [SMILES: CC(C)CCC(=O)Nc1cccc(COc2nc3c(Cl)cccc3n2-c2nnnn2C)n1] |
| 10 | E | G2 | 30%^{c} | δ 8.19 (d, J = 8.3 Hz, 1H), 7.97 (br s, 1H), 7.71 (t, J = 7.9 Hz, 1H), 7.35 (dd, J = 6.8, 2.2 Hz, 1H), 7.24-7.17 (m, 2H), 7.08 (d, J = 7.4 Hz, 1H), 5.69 (s, 2H), 4.04 (s, 3H), 2.78 (p, J = 8.1 Hz, 1H), 1.98 (ddd, J = 11.7, 7.3, 6.0 Hz, 2H), 1.88 (dq, J = 12.4, 7.9 Hz, 2H), 1.84-1.72 (m, 2H), 1.70-1.61 (m, 2H) | 3.96 (B) | 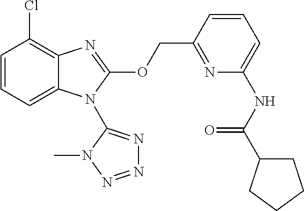 [SMILES: Cn1nnnc1-n1c(OCc2cccc(NC(=O)C3CCCC3)n2)nc2c(Cl)cccc21] |
| 11 | E | G3 | 70% | δ 7.97 (d, J = 8.4 Hz, 1H), 7.76-7.69 (m, 1H), 7.39 (s, 1H), 7.34 (dd, J = 7.5, 1.5 Hz, 1H), 7.22 (dd, J = 8.1, 1.5 Hz, 1H), 7.21-7.16 (m, 1H), 7.10 (d, J = 7.3 Hz, 1H), 5.97 (ddt, J = 17.1, 10.5, 5.7 Hz, 1H), 5.67 (s, 2H), 5.38 (dq, J = 17.2, 1.5 Hz, 1H), 5.28 (ddd, J = 10.5, 2.5, 1.2 Hz, 1H), 4.69 (dt, J = 5.7, 1.4 Hz, 2H), 4.05 (s, 3H) | 3.48 (B) | 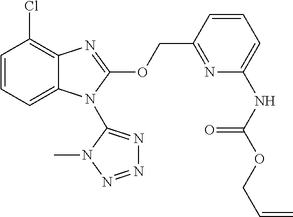 [SMILES: C=CCOC(=O)Nc1cccc(COc2nc3c(Cl)cccc3n2-c2nnnn2C)n1] |
| 12 | E | G4 | 46%^{d} | δ 8.19 (d, J = 8.4 Hz, 1H), 7.95 (s, 1H), 7.71 (t, J = 7.9 Hz, 1H), 7.35 (dd, J = 6.7, 2.3 Hz, 1H), 7.22-7.18 (m, 2H), 7.09 (d, J = 7.4 Hz, 1H), 5.69 (s, 2H), 4.04 (s, 3H), 2.41 (t, J = 7.4 Hz, 2H), 1.82-1.72 (m, 2H), 1.01 (t, J = 7.4 Hz, 3H) | 3.32 (B) | 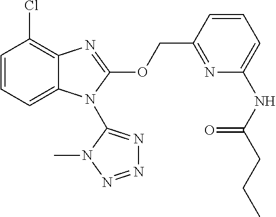 [SMILES: CCCC(=O)Nc1cccc(COc2nc3c(Cl)cccc3n2-c2nnnn2C)n1] |
| 13 | E | [2138815-81-1]^{b} | 41%^{d} | δ 7.98 (d, J = 8.4 Hz, 1H), 7.75-7.70 (m, 1H), 7.40-7.30 (br m, 2H), 7.24-7.16 (m, 2H), 7.09 (d, J = 7.3 Hz, 1H), 5.67 (s, 2H), 4.20 (dd, J = 8.7, 4.7 Hz, 2H), 4.04 (s, 3H), 1.72- 1.64 (m, 2H), 1.42 (p, J = 7.4 Hz, 2H), 0.96 (t, J = 7.4 Hz, 3H) | 3.84 (B) | 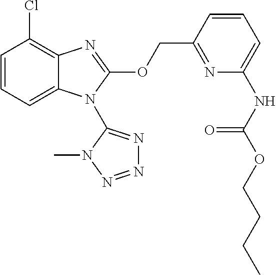 [SMILES: CCCCOC(=O)Nc1cccc(COc2nc3c(Cl)cccc3n2-c2nnnn2C)n1] |
| 14 | E | G5 | 45%^{d} | δ 7.97 (d, J = 8.4 Hz, 1H), 7.72 (t, J = 7.9 Hz, 1H), 7.34 (dd, J = 7.5, 1.5 Hz, 1H), 7.31 (s, 1H), 7.24-7.16 (m, 2H), 7.09 (d, J = 7.4 Hz, 1H), 5.66 (s, 2H), 4.25 (q, J = 7.2 Hz, 2H), 4.04 (s, 3H), 1.33 (t, J = 7.1 Hz, 3H) | 3.62 (A) | 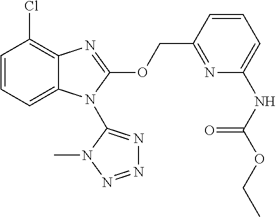 [SMILES: CCOC(=O)Nc1cccc(COc2nc3c(Cl)cccc3n2-c2nnnn2C)n1] |
| 15 | E | G6 | 24%^{d} | δ 7.97 (d, J = 8.4 Hz, 1H), 7.75-7.69 (m, 1H), 7.35 (dd, J = 7.4, 1.5 Hz, 1H), 7.32 (s, 1H), 7.24-7.17 (m, 2H), 7.09 (d, J = 7.4 Hz, 1H), 5.67 (s, 2H), 4.04 (s, 3H), 3.98 (d, J = 6.6 Hz, 2H), 2.00 (dt, J = 13.4, 6.7 Hz, 1H), 0.98 (d, J = 6.6.Hz, 6H) | 4.01 (A) | 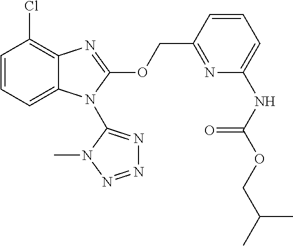 [SMILES: CC(C)COC(=O)Nc1cccc(COc2nc3c(Cl)cccc3n2-c2nnnn2C)n1] |
| 16 | E | G7 | 33%^{d} | δ 7.97 (d, J = 8.4 Hz, 1H), 7.75-7.68 (m, 1H), 7.35 (dd, J = 7.5, 1.5 Hz, 1H), 7.30 (s, 1H), 7.24-7.16 (m, 2H), 7.09 (d, J = 7.4 Hz, 1H), 5.66 (s, 2H), 4.15 (t, J = 6.7 Hz, 2H), 4.04 (s, 3H), 1.76-1.67 (m, 2H), 0.99 (t, J = 7.4 Hz, 3H) | 3.84 (A) | 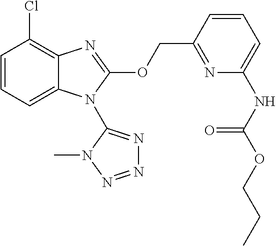 [SMILES: CCCOC(=O)Nc1cccc(COc2nc3c(Cl)cccc3n2-c2nnnn2C)n1] |
| 17 | E | G8 | 40%^{d} | δ 7.97 (d, J = 8.4 Hz, 1H), 7.74-7.68 (m, 1H), 7.35 (dd, J = 7.6, 1.3 Hz, 1H), 7.24-7.16 (m, 3H), 7.08 (d, J = 7.3 Hz, 1H), 5.65 (s, 2H), 5.22 (td, J = 6.0, 3.1 Hz, 1H), 4.03 (s, 3H), 1.95-1.84 (m, 2H), 1.77 (dd, J = 13.2, 9.0 Hz, 4H), 1.66-1.59 (m, 2H) | 4.09 (A) | 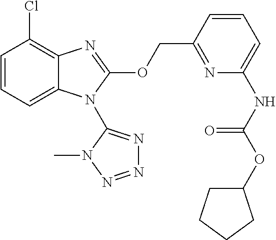 [SMILES: Cn1nnnc1-n1c(OCc2cccc(NC(=O)OC3CCCC3)n2)nc2c(Cl)cccc21] |
| 18 | E | G9 | 8%^{d} | δ 7.97 (d, J = 8.4 Hz, 1H), 7.74-7.68 (m, 1H), 7.35 (dd, J = 7.6, 1.3 Hz, 1H), 7.25-7.16 (m, 3H), 7.08 (d, J = 7.4 Hz, 1H), 5.66 (s, 2H), 5.07-5.00 (m, 1H), 4.04 (s, 3H), 1.32 (d, J = 6.6 Hz, 6H) | 3.70 (B) | 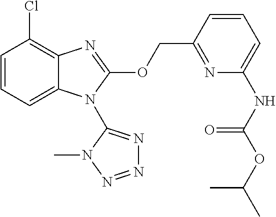 [SMILES: CC(C)OC(=O)Nc1cccc(COc2nc3c(Cl)cccc3n2-c2nnnn2C)n1] |
| 19 | E | G10 | 55%^{d} | δ 8.27 (d, J = 7.8 Hz, 1H), 8.12 (br s, 1H), 7.76 (t, J = 7.8 Hz, 1H), 7.37 (dd, J = 6.3, 2.7 Hz, 1H), 7.24-7.20 (m, 2H), 7.14 (d, J = 7.4 Hz, 1H), 5.72 (s, 2H), 4.08 (s, 3H), 1.36 (s, 9H) | 3.76 (A) | 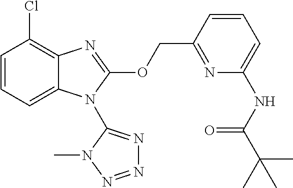 [SMILES: Cn1nnnc1-n1c(OCc2cccc(NC(=O)C(C)(C)C)n2)nc2c(Cl)cccc21] |
| 20 | E4 | [148167-30-0]^{b} | 30% | δ 8.88 (s, 1H), 8.27 (d, J = 8.2 Hz, 1H), 7.77 (t, J = 7.9 Hz, 1H), 7.36 (dd, J = 8.7, 7.4 Hz, 2H), 7.21 (d, J = 7.4 Hz, 1H), 7.16-7.11 (m, 2H), 7.10-7.05 (m, 2H), 7.02 (dd, J = 8.8, 0.9 Hz, 2H), 5.65 (s, 2H), 4.65 (s, 2H), 3.99 (s, 3H), 2.59 (s, 3H) | 3.82 (B) | 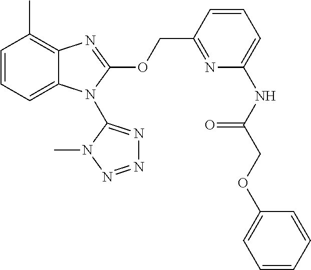 [SMILES: Cc1cccc2c1nc(OCc1cccc(NC(=O)COc3ccccc3)n1)n2-c1nnnn1C] |
| 21 | E4 | [203321-83-9]^{b} | 48% | δ 7.95 (d, J = 8.7 Hz, 1H), 7.70 (t, J = 8.0 Hz, 1H), 7.25 (br s, 1H), 7.16-7.05 (m, 4H), 5.60 (s, 2H), 4.02 (s, 3H), 2.59 (s, 3H), 1.53 (s, 9H) | 3.68 (B) | 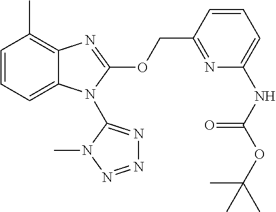 [SMILES: Cc1cccc2c1nc(OCc1cccc(NC(=O)OC(C)(C)C)n1)n2-c1nnnn1C] |

^{a}RT = LCMS retention time in minutes using indicated Method (A-D);
^{b}CAS Registry No. Compound prepared as described in WO2017/178819;
^{c}Recrystallised from EtOAc/PE (71% before recrystallisation);
^{d}The alcohol was deprotonated first at 0° C. for 15 min then Ar X was added.

### Example 22—tert-Butyl (6-(((4-hydroxy-1-(1-methyl-1H-tetrazol-5-yl)-1H-benzo[d]imidazol-2-yl)oxy)methyl)pyridin-2-yl)carbamate 22

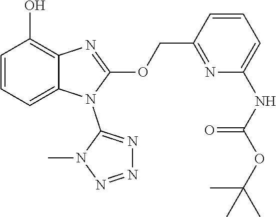 [SMILES: Cn1nnnc1-n1c(OCc2cccc(NC(=O)OC(C)(C)C)n2)nc2c(O)cccc21]

A solution of tert-butyl (6-(((4-(allyloxy)-1-(1-methyl-1H-tetrazol-5-yl)-1H-benzo[d]imidazol-2-yl)oxy)methyl)pyridin-2-yl)carbamate 7 (21 mg, 0.044 mmol) and 2,2-dimethyl-1,3-dioxane-4,6-dione (12.6 mg, 0.088 mmol) in dry DMF (1 mL) was deoxygenated (4 cycles of vacuum/N₂), treated with tetrakis(triphenylphosphine)palladium (5.1 mg, 4.4 μmol), deoxygenated again and stirred at RT for 2 h. Water (10 mL) was added and the mixture was extracted with EtOAc (10 mL). The organics were washed with water (3×10 mL) and brine, dried (MgSO₄) and chromatographed on silica (5 g cartridge) eluting with 10-50% EtOAc/DCM to give tert-butyl(6-(((4-hydroxy-1-(1-methyl-1H-tetrazol-5-yl)-1H-benzo[d]imidazol-2-yl)oxy)methyl)pyridin-2-yl)carbamate 22 (11 mg, 57%) as an off-white solid.

¹H NMR (500 MHz, CDCl₃) δ 7.93 (d, J=8.4 Hz, 1H), 7.73-7.63 (m, 1H), 7.57 (br s, 1H), 7.132 (br s, 1H), 7.13 (t, J=8.1 Hz, 1H), 7.00 (d, J=7.3 Hz, 1H), 6.85 (dd, J=8.2, 0.8 Hz, 1H), 6.81 (dd, J=8.1, 0.8 Hz, 1H), 5.59 (s, 2H), 4.01 (s, 3H), 1.51 (s, 9H); LCMS (Method B): 3.15 min (439.1, MH⁺).

### Example 23—N-(6-(((4-Chloro-1-(1-methyl-1H-tetrazol-5-yl)-1H-benzo[d]imidazol-2-yl)oxy)methyl)pyridin-2-yl)propionamide 23

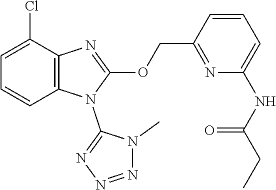 [SMILES: CCC(=O)Nc1cccc(COc2nc3c(Cl)cccc3n2-c2nnnn2C)n1]

A stirred suspension of 6-(((4-chloro-1-(1-methyl-1H-tetrazol-5-yl)-1H-benzo[d]imidazol-2-yl)oxy)methyl)pyridin-2-amine H (26.2 mg, 0.073 mmol) in dry DCM (1 mL) was cooled with ice/water and treated with triethylamine (11 μL, 0.081 mmol) then propionyl chloride (6.5 μL, 0.074 mmol). After 40 min the mixture was allowed to warm to RT for 20 min then was re-cooled and treated with more triethylamine (5.12 μL, 0.037 mmol) and propionyl chloride (3.2 μL, 0.037 mmol). After 25 min the solution was allowed to warm to RT for 5 min, transferred to a silica cartridge (4 g Claricep), chromatographed eluting with 30-70% EtOAc/PE and triturated with DCM/PE to give N-(6-(((4-chloro-1-(1-methyl-1H-tetrazol-5-yl)-1H-benzo[d]imidazol-2-yl)oxy)methyl)pyridin-2-yl)propionamide 23 (20 mg, 66%) as a white solid.

¹H NMR (500 MHz, CDCl₃) δ 8.22 (br s, 1H), 8.19 (d, J=8.4 Hz, 1H), 7.71 (t, J=7.9 Hz, 1H), 7.34 (dd, J=6.9, 2.1 Hz, 1H), 7.23-7.16 (m, 2H), 7.09 (d, J=7.3 Hz, 1H), 5.68 (s, 2H), 4.03 (s, 3H), 2.46 (q, J=7.5 Hz, 2H), 1.24 (t, J=7.5 Hz, 3H); LCMS (Method B): 3.15 min (413.1, MH⁺).

Following the general procedure of Example 23, using the appropriate starting materials (X is a leaving group such as chloro or OCOR, adding further reagents as required (usually 0.1-1 extra molar equivalents), triturating (e.g. with ether or DCM/PE) if required after chromatography to provide a solid, there were thus obtained the following Examples (Ex. 24-33):

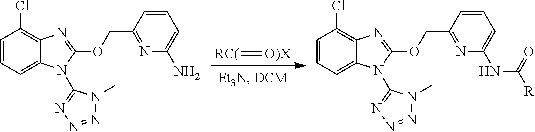

| Ex. | RC(═O)X | Yield | ¹H NMR δ (CDCl₃) | RT^{a} | Structure |
|---|---|---|---|---|---|
| 24 | 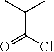 | 69% | δ 8.25 (br s, 1H), 8.21 (d, J = 8.4 Hz, 1H), 7.76-7.68 (m, 1H), 7.34 (dd, J = 6.9, 2.1 Hz, 1H), 7.23-7.16 (m, 2H), 7.10 (d, J = 7.4 Hz, 1H), 5.69 (s, 2H), 4.04 (s, 3H), 2.67-2.56 (m, 1H), 1.26 (d, J = | 3.34 (B) | 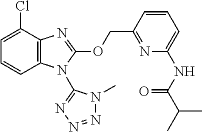 [SMILES: CC(C)C(=O)Nc1cccc(COc2nc3c(Cl)cccc3n2-c2nnnn2C)n1] |
| | | | 6.9 Hz, 6H) | | |
| 25 | 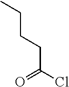 | 43% | δ 8.52 (s, 1H), 8.22 (d, J = 8.4 Hz, 1H), 7.75- 7.69 (m, 1H), 7.34 (dd, J = 6.9, 2.1 Hz, 1H), 7.23-7.15 (m, 2H), 7.11 (d, J = 7.4 Hz, 1H), 5.67 (s, 2H), 4.03 (s, 3H), 2.48-2.39 (m, 2H), 1.79-1.64 (m, 2H), 1.44-1.36 (m, | 3.57 (B) | 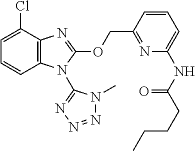 [SMILES: CCCCC(=O)Nc1cccc(COc2nc3c(Cl)cccc3n2-c2nnnn2C)n1] |
| | | | 2H), 0.94 (t, J = 7.4 Hz, | | |
| | | | 3H) | | |
| 26 | 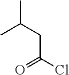 | 68% | δ 8.24 (s, 1H), 8.21 (d, J = 8.4 Hz, 1H), 7.75- 7.67 (m, 1H), 7.34 (dd, J = 7.0, 2.0 Hz, 1H), 7.23-7.16 (m, 2H), 7.10 (d, J = 7.3 Hz, 1H), 5.68 (s, 2H), 4.03 (s, 3H), 2.29 (d, J = 6.9 Hz, 2H), 2.25-2.14 (m, 1H), 1.01 (d, J = | 3.45 (B) | 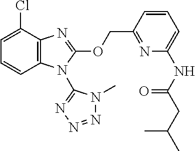 [SMILES: CC(C)CC(=O)Nc1cccc(COc2nc3c(Cl)cccc3n2-c2nnnn2C)n1] |
| | | | 6.6 Hz, 6H) | | |
| 27 | 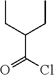 | 41% | δ 8.25 (d, J = 8.3 Hz, 1H), 8.19 (br s, 1H), 7.72 (t, J = 7.9 Hz, 1H), 7.34 (dd, J = 7.0, 2.0 Hz, 1H), 7.24-7.15 (m, 2H), 7.11 (d, J = 7.4 Hz, 1H), 5.69 (s, 2H), 4.04 (s, 3H), 2.17 (ddd, J = 14.2, 8.9, 5.3 Hz, 1H), 1.77-1.67 (m, | 3.67 (B) | 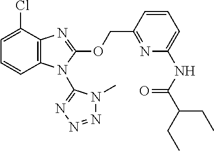 [SMILES: CCC(CC)C(=O)Nc1cccc(COc2nc3c(Cl)cccc3n2-c2nnnn2C)n1] |
| | | | 2H), 1.59 (ddd, J = | | |
| | | | 13.6, 10.1, 6.4 Hz, | | |
| | | | 2H), 0.94 (t, J = 7.4 Hz, | | |
| | | | 6H) | | |
| 28 | 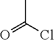 | 60%^{b} | δ 8.46 (br s, 1H), 8.18 (d, J = 8.3 Hz, 1H), 7.76-7.67 (m, 1H), 7.34 (dd, J = 6.5, 2.5 Hz, 1H), 7.23-7.15 (m, 2H), 7.10 (d, J = 7.2 Hz, 1H), 5.67 (s, 2H), 4.04 (s, 3H), 2.22 (s, 3H) | 2.95 (B) | 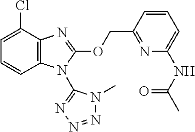 [SMILES: CC(=O)Nc1cccc(COc2nc3c(Cl)cccc3n2-c2nnnn2C)n1] |
| 29 | 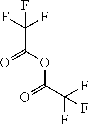 [SMILES: O=C(OC(=O)C(F)(F)F)C(F)(F)F] | 65%^{b,c} | δ 8.76 (br s, 1H), 8.15 (d, J = 8.2 Hz, 1H), 7.82 (t, J = 7.9 Hz, 1H), 7.35 (dd, J = 7.6, 1.4 Hz, 1H), 7.25 (d, J = 7.9 Hz, 1H), 7.23-7.14 (m, 2H), 5.73 (s, 2H), 4.06 (s, 3H) | 3.49 (B) | 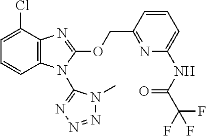 [SMILES: Cn1nnnc1-n1c(OCc2cccc(NC(=O)C(F)(F)F)n2)nc2c(Cl)cccc21] |
| 30 | 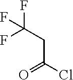 | 57%^{b,d} | δ 8.48 (br s, 1H), 8.09 (d, J = 8.2 Hz, 1H), 7.72 (t, J = 7.9 Hz, 1H), 7.33 (dt, J = 8.1, 4.0 Hz, 1H), 7.22-7.15 (m, 2H), 7.11 (dd, J = 9.7, 2.2 Hz, 1H), 5.70 (s, 2H), 4.07 (s, 3H), 3.33 (q, J = 10.2 Hz, 2H) | 3.49 (B) | 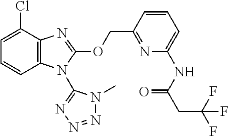 [SMILES: Cn1nnnc1-n1c(OCc2cccc(NC(=O)CC(F)(F)F)n2)nc2c(Cl)cccc21] |
| 31 | 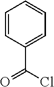 | 23%^{e,f,g} | δ 8.69 (br s, 1H), 8.39 (d, J = 8.3 Hz, 1H), 7.97 (d, J = 7.2 Hz, 2H), 7.80 (t, J = 7.9 Hz, 1H), 7.59 (t, J = 7.4 Hz, 1H), 7.53 (t, J = 7.5 Hz, 2H), 7.34 (dt, J = 8.1, 4.1 Hz, 1H), 7.23-7.15 (m, 3H), 5.73 (s, 2H), 4.05 (s, 3H) | 1.99 (D) | 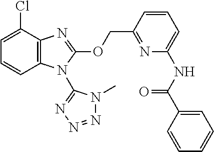 [SMILES: Cn1nnnc1-n1c(OCc2cccc(NC(=O)c3ccccc3)n2)nc2c(Cl)cccc21] |
| 32 | 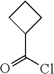 | 21%^{e,h} | δ 8.36-8.26 (m, 1H), 8.27-8.20 (m, 1H), 7.78-7.66 (m, 1H), 7.36-7.31 (m, H), 7.20 (s, 2H), 7.13-7.07 (m, 1H), 5.68 (s, 2H), 4.03 (s, 3H), 3.32-3.21 (m, 1H), 2.47-2.33 (m, 2H), 2.32-2.16 (m, | 3.29 (B) | 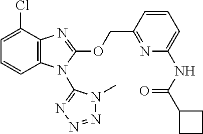 [SMILES: Cn1nnnc1-n1c(OCc2cccc(NC(=O)C3CCC3)n2)nc2c(Cl)cccc21] |
| | | | 4H), 1.97-1.84 (m, 2H) | | |
| 33 | 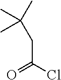 | 51%^{e,f,g} | δ 8.23 (s, 1H), 8.21 (d, J = 8.4 Hz, 1H), 7.71 (t, J = 7.9 Hz, 1H), 7.33 (dd, J = 7.0, 2.0 Hz, 1H), 7.22-7.16 (m, 2H), 7.09 (d, J = 7.5 Hz, 1H), 5.68 (s, 2H), 4.03 (s, 3H), 2.29 (s, 2H), 1.08 (s, 9H) | 3.85 (B) | 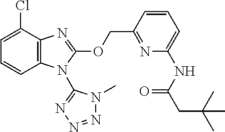 [SMILES: Cn1nnnc1-n1c(OCc2cccc(NC(=O)CC(C)(C)C)n2)nc2c(Cl)cccc21] |

^{a}RT = LCMS retention time in minutes using indicated Method (A-D);
^{b}Pyridine was used as base instead of triethylamine;
^{c}A total of 3.6 equivalents of (CF₃C(═O))₂O was added in three aliquots, preceded on the first two occasions by pyridine (2 × 1.4 equiv.);
^{d}An ice-cooled solution of 3,3,3-trifluoropropionic acid (52 μL, 0.59 mmol) in dry DCM (1.0 mL) was treated with oxalyl chloride (50 μL, 0.58 mmol) and 1 drop of dry DMF then warmed to RT for 30 min and an aliquot was used directly (ca. 1.1 equiv.) as an 0.6 M DCM solution of 3,3,3-trifluoropropanoyl chloride (assuming quantitative conversion);
^{e}Reaction carried out at RT;
^{f}DMAP was added during the reaction;
^{g}Aqueous work-up including base-wash (1M aq. NaOH) was conducted before chromatography;
^{h}DMAP was used in place of triethylamine.

### Example 34—tert-Butyl (6-(((4-bromo-1-(1-methyl-1H-tetrazol-5-yl)-1H-benzo[d]imidazol-2-yl)oxy)methyl)pyridin-2-yl)carbamate 34

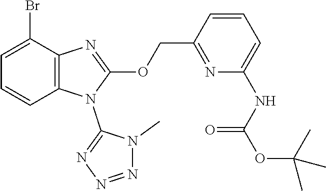 [SMILES: Cn1nnnc1-n1c(OCc2cccc(NC(=O)OC(C)(C)C)n2)nc2c(Br)cccc21]

A solution of tert-butyl (6-(hydroxymethyl)pyridin-2-yl)carbamate (167 mg, 0.74 mmol) and 4-bromo-1-(1-methyl-1H-tetrazol-5-yl)-2-(methylsulfonyl)-1H-benzo[d]imidazole E5 (260 mg, 0.73 mmol) in dry DMF (3 mL) was treated with a solution of LHMDS (1.5 mL of a 1M solution in THF, 1.5 mmol) and stirred for 20 min then saturated aqueous ammonium chloride solution (3 mL) was added. The mixture was diluted with EtOAc (30 mL), washed with water (4×30 mL) and brine, dried (MgSO₄), chromatographed on silica (20 g Claricep cartridge) eluting with 30-45% EtOAc/PE and triturated with ether to give tert-butyl (6-(((4-bromo-1-(1-methyl-1H-tetrazol-5-yl)-1H-benzo[d]imidazol-2-yl)oxy)methyl)pyridin-2-yl)carbamate 34 (309 mg, 85%) as a white solid.

¹H NMR (500 MHz, CDCl₃) δ 7.97 (d, J=8.4 Hz, 1H), 7.71 (t, J=7.9 Hz, 1H), 7.50 (dd, J=8.0, 0.9 Hz, 1H), 7.33 (s, 1H), 7.26-7.24 (m, 2H), 7.13 (t, J=8.0 Hz, 1H), 7.09 (d, J=7.4 Hz, 1H), 5.66 (s, 2H), 4.04 (s, 3H), 1.53 (s, 9H); LCMS (Method D): 2.18 min (502.9, MH⁺ for one of a pair of Br isotopes).

Following the general procedure of Example 34, using the appropriate starting materials (X is a leaving group such as halo, methylsulfonyl, arylsulfonyl), and with chromatography in a suitable solvent system (typically EtOAc/DCM or EtOAc/PE) followed by trituration (e.g. with ether or DCM/PE) if required, there were thus obtained the following Examples (Ex. 35-42):

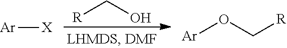

| Ex. | Ar—X | 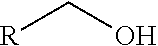 | Yield | ¹H NMR δ (CDCl₃) | RT^{a} | 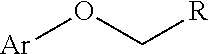 |
|---|---|---|---|---|---|---|
| 35 | E5 | [2138815-81-1] | 84% | δ 8.03 (d, J = 8.4 Hz, 1H), 7.76 (t, J = 7.9 Hz, 1H), 7.67 (s, 1H), 7.50 (dd, J = 8.0, 0.9 Hz, 1H), 7.24 (dd, J = 8.0, 0.8 Hz, 1H), 7.17-7.10 (m, 2H), 5.68 (s, 2H), 4.20 (t, J = 6.7 Hz, 2H), 4.05 (s, 3H), 1.70-1.65 (m, 2H), 1.47-1.38 (m, 2H), 0.95 (t, J = 7.4 Hz, 3H). | 2.19 (D) | 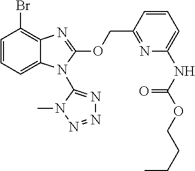 [SMILES: CCCCOC(=O)Nc1cccc(COc2nc3c(Br)cccc3n2-c2nnnn2C)n1] |
| 36 | E5 | [148167-30-0] | 70% | δ 8.88 (s, 1H), 8.28 (d, J = 8.3 Hz, 1H), 7.79 (t, J = 7.9 Hz, 1H), 7.50 (d, J = 8.0 Hz, 1H), 7.36 (t, J = 7.9 Hz, 2H), 7.26- 7.20 (m, 2H), 7.13 (t, J = 8.0 Hz, 1H), 7.07 (t, J = 7.4 Hz, 1H), 7.02 (d, J = 8.2 Hz, 2H), 5.70 (s, 2H), 4.65 (s, 2H), 3.99 (s, 3H) | 2.11 (D) | 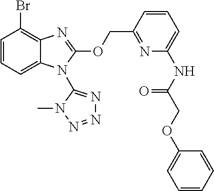 [SMILES: Cn1nnnc1-n1c(OCc2cccc(NC(=O)COc3ccccc3)n2)nc2c(Br)cccc21] |
| 37 | E5 | G2 | 60% | δ 8.19 (d, J = 8.3 Hz, 1H), 8.03 (s, 1H), 7.70 (t, J = 7.9 Hz, 1H), 7.49 (dd, J = 8.0, 0.9 Hz, 1H), 7.24 (dd, J =8 .1, 0.9 Hz, 1H), 7.17-7.07 (m, 2H), 5.68 (s, 2H), 4.04 (s, 3H), 2.77 (p, J = 8.1 Hz, 1H), 2.01-1.93 (m, 2H), 1.92-1.83 (m, 2H), 1.82-1.73 (m, 2H), 1.68-1.58 (m, 2H) | 3.89 (B) | 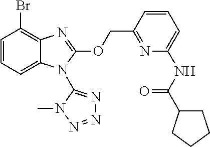 [SMILES: Cn1nnnc1-n1c(OCc2cccc(NC(=O)C3CCCC3)n2)nc2c(Br)cccc21] |
| 38 | E5 | G6 | 85% | δ 8.00 (d, J = 8.4 Hz, 1H), 7.74 (t, J = 7.9 Hz, 1H), 7.55 (br s, 1H), 7.50 (dd, J = 8.0, 0.9 Hz, 1H), 7.25 (dd, J = 8.1, 0.9 Hz, 1H), 7.16-7.10 (m, 2H), 5.68 (s, 2H), 4.04 (s, 3H), 3.98 (d, J = 6.7 Hz, 2H), 2.00-1.95 (m, 1H), 0.97 (d, J = 6.7 Hz, 6H). | 2.19 (D) | 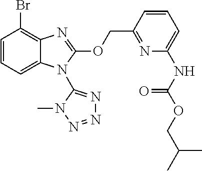 [SMILES: CC(C)COC(=O)Nc1cccc(COc2nc3c(Br)cccc3n2-c2nnnn2C)n1] |
| 39 | E5 | G7 | 67% | δ 7.98 (d, J = 8.5 Hz, 1H), 7.72 (t, J = 7.9 Hz, 1H), 7.50 (dd, J = 8.0, 0.8 Hz, 1H), 7.40 (s, 1H), 7.26-7.22 (m, 1H), 7.17- 7.08 (m, 2H), 5.67 (s, 2H), 4.15 (t, J = 6.7 Hz, 2H), 4.04 (s, 3H), 1.77-1.67 (m, 2H), 0.98 (t, J = 7.4 Hz, 3H) | 4.05 (B) | 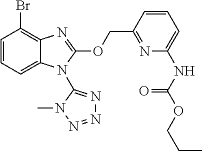 [SMILES: CCCOC(=O)Nc1cccc(COc2nc3c(Br)cccc3n2-c2nnnn2C)n1] |
| 40 | E5 | G8 | 44% | δ 7.98 (d, J = 8.4 Hz, 1H), 7.76- 7.68 (m, 1H), 7.50 (dd, J = 8.0, 0.9 Hz, 1H), 7.33 (s, 1H), 7.26 (dd, J = 8.0, 0.9 Hz, 1H), 7.13 (t, J = 8.0 Hz, 1H), 7.10 (d, J = 7.3 Hz, 1H), 5.66 (s, 2H), 5.29-5.17 (m, 1H), 4.03 (s, 3H), 1.97-1.83 (m, 2H), 1.83-1.71 (m, 4H), 1.68- 1.49 (m, 4H) | 4.23 (B) | 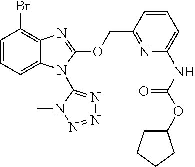 [SMILES: Cn1nnnc1-n1c(OCc2cccc(NC(=O)OC3CCCC3)n2)nc2c(Br)cccc21] |
| 41 | E5 | G11 | 63%^{b} | δ 8.20 (d, J = 8.3 Hz, 1H), 7.97 (s, 1H), 7.72 (t, J = 7.9 Hz, 1H), 7.51 (d, J = 8.0 Hz, 1H), 7.24 (s, 1H), 7.18-7.07 (m, 2H), 5.69 (s, 2H), 4.04 (s, 3H), 2.29 (d, J = 7.1 Hz, 2H), 2.26- 2.17 (m, 1H), 1.02 (d, J = 6.5 Hz, 6H) | 3.77 (B) | 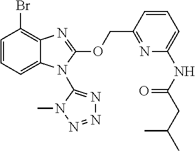 [SMILES: CC(C)CC(=O)Nc1cccc(COc2nc3c(Br)cccc3n2-c2nnnn2C)n1] |
| 42 | E5 | G12 | 68% | (DMSO-d6) δ 10.49 (s, 1H), 8.08 (d, J = 8.4 Hz, 1H), 7.87- 7.76 (m, 1H), 7.54 (dd, J = 8.0, 0.9 Hz, 1H), 7.39 (dd, J = 8.0, 0.9 Hz, 1H), 7.22 (d, J = 7.2 Hz, 1H), 7.19 (t, J = 8.0 Hz, 1H), 5.66 (s, 2H), 4.08 (s, 3H), 2.38 (t, J = 7.4 Hz, 2H), 1.59- 1.50 (m, 2H), 1.34-1.25 (m, 2H), 0.88 (t, J = 7.4 Hz, 3H). | 2.07 (D) | 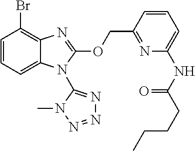 [SMILES: CCCCC(=O)Nc1cccc(COc2nc3c(Br)cccc3n2-c2nnnn2C)n1] |

^{a}RT = LCMS retention time in minutes using indicated Method (A-D);
^{b}NaHMDS (2M in THF) was used at RT instead of LHMDS at 0° C.

Following the general procedure of Example 1, using the appropriate starting materials and with chromatography in a suitable solvent system (typically EtOAc/DCM or EtOAc/PE) followed by trituration (e.g. with ether or DCM/PE) if required, there were thus obtained the following Examples (Ex. 43-44):

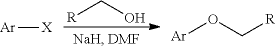

| Ex. | Ar—X | 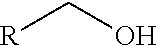 | Yield | ¹H NMR δ (CDCl₃) | RT^{a} | 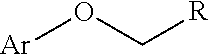 |
|---|---|---|---|---|---|---|
| 43 | E | G13 | 17%^{b} | δ 7.31 (dd, J = 8.0, 1.1 Hz, 1H), 7.17 (dd, J = 8.2, 1.1 Hz, 1H), 7.09 (t, J = 8.1 Hz, 1H), 6.79 (s, 1H), 5.49 (s, J = 0.8 Hz, 2H), 4.18 (s, 3H), 2.43- 2.38 (m, 2H), 1.68 (dt, J = 20.8, 7.5 Hz, 2H), 1.40-1.32 (m, 2H), 0.91 (t, J = 7.4 Hz, 3H) | 3.62 (B) | 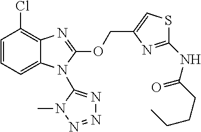 [SMILES: CCCCC(=O)Nc1nc(COc2nc3c(Cl)cccc3n2-c2nnnn2C)cs1] |
| 44 | E | G14 | 21%^{c} | δ 9.28 (s, 1H), 7.33 (dd, J = 7.4, 1.6 Hz, 1H), 7.21-7.17 (m, 2H), 7.11 (s, 1H), 5.67 (s, 2H), 3.98 (s, 3H), 2.49-2.44 (m, 2H), 1.65-1.60 (m, 3H), 0.92 (d, J = 6.4 Hz, 6H) | 4.05 (B) | 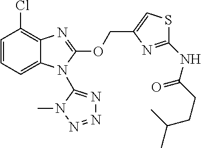 [SMILES: CC(C)CCC(=O)Nc1nc(COc2nc3c(Cl)cccc3n2-c2nnnn2C)cs1] |

^{a}RT = LCMS retention time in minutes using indicated Method (A-D);
^{b}1.4 molar eq. of NaH were used;
^{c}2.1 molar eq. of NaH were used.

### Example 45 tert-Buty (6-(((1-(1-methyl-1H-tetrazol-5-yl)-4-phenyl-1H-benzo[d]imidazol-2-yl)oxy)methyl)pyridin-2-yl)carbamate 45

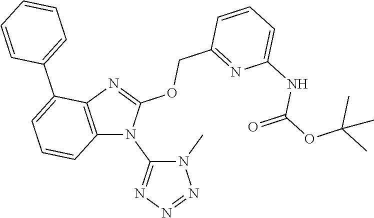 [SMILES: Cn1nnnc1-n1c(OCc2cccc(NC(=O)OC(C)(C)C)n2)nc2c(-c3ccccc3)cccc21]

A solution of tert-butyl (6-(((4-bromo-1-(1-methyl-1H-tetrazol-5-yl)-1H-benzo[d]imidazol-2-yl)oxy)methyl)pyridin-2-yl)carbamate 34 (21 mg, 0.042 mmol) and phenylboronic acid (10 mg, 0.082 mmol) in 1,4-dioxane (2 mL) was treated with potassium carbonate (17 mg, 0.13 mmol) and water (0.5 mL), deoxygenated with a stream of nitrogen for 5 min, then treated with [1,1′-bis(diphenylphosphino)ferrocene]dichloropalladium(II), complex with dichloromethane (3 mg, 0.004 mmol) and subjected to microwave irradiation at 100° C. for 30 min. The mixture was diluted with water (10 mL) and extracted with EtOAc (2×10 mL). Organics were washed with water (2×10 mL) and brine then dried (MgSO₄) and chromatographed on silica (12 g Claricep cartridge) eluting with 20-40% EtOAc/PE to give tert-butyl (6-(((1-(1-methyl-1H-tetrazol-5-yl)-4-phenyl-1H-benzo[d]imidazol-2-yl)oxy)methyl) pyridin-2-yl)carbamate 45 (21 mg, 100%) as a colourless foam.

¹H NMR (500 MHz, CDCl₃) δ 8.02 (d, J=8.6 Hz, 1H), 7.94 (dd, J=8.2, 1.1 Hz, 2H), 7.75 (t, J=8.0 Hz, 1H), 7.52-7.48 (m, 3H), 7.42-7.38 (m, 1H), 7.32 (t, J=7.9 Hz, 1H), 7.27-7.22 (m, 1H), 7.12 (d, J=7.4 Hz, 1H), 5.62 (s, 2H), 4.08 (s, 3H), 1.53 (s, 9H); LCMS (Method D): 2.34 min (499.1, MH⁺).

Following the general procedure of Example 45, using the appropriate starting materials (X is typically a halide or triflate) there were thus obtained the following Examples (Ex. 46-48):

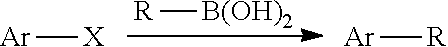

| Ex. | Ar—X | RB(OH)₂ | Yield | ¹H NMR δ (CDCl₃) | RT^{a} | Ar—R |
|---|---|---|---|---|---|---|
| 46 | Ex. 38 | 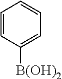 | 73% | δ 7.97 (d, J = 8.4 Hz, 1H), 7.95-7.91 (m, 2H), 7.73 (t, J = 7.9 Hz, 1H), 7.52-7.47 (m, 3H), 7.42-7.38 (m, 1H), 7.32 (t, J = 7.9 Hz, 1H), 7.25 (dd, J = 8.0, 1.1 Hz, 1H), 7.11 (d, J = 7.4 Hz, 1H), 5.60 (s, 2H), 4.06 (s, 3H), 3.97 (d, J = 6.7 Hz, 2H), 2.03-1.95 (m, 1H), 0.97 (d, J = 6.7 Hz, 6H) | 2.42 (D) | 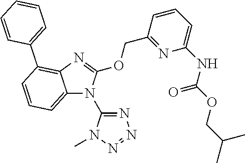 |
| 47 | Ex. 38 | 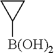 | 44%^{b} | δ 8.00 (d, J = 8.3 Hz, 1H), 7.74 (t, J = 7.9 Hz, 1H), 7.61 (br s, 1H), 7.18-7.10 (m, 2H), 7.02 (dd, J = 8.0, 0.9 Hz, 1H), 6.82 (d, J = 7.7 Hz, 1H), 5.62 (s, 2H), 4.03 (s, 3H), 3.98 (d, J = 6.7 Hz, 2H), 2.52-2.43 (m, 1H), 2.04-1.95 (m, 1H), 1.11-1.06 (m, 2H), 1.02-0.98 (m, 2H), 0.98 (t, | 2.35 (D) | 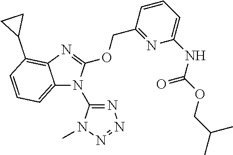 |
| | | | | J = 5.7 Hz, 6H). | | |
| 48 | Ex. 34 | 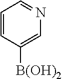 | 36%^{b} | δ 9.32 (s, 1H), 8.64 (s, 1H), 8.37 (d, J = 7.2 Hz, 1H), 7.94 (d, J =8.4 Hz, 1H), 7.73 (t, J = 7.8 Hz, 1H), 7.52 (br d, J = 6.0 Hz, 2H), 7.38-7.33 (m, 3H), 7.10 (d, J = 7.2 Hz, 1H), 5.59 (s, 2H), 4.08 (s, 3H), 1.52 (s, 9H). | 2.22 (B) | 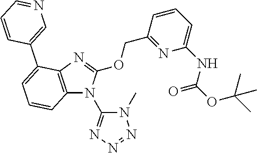 |

^{a}RT = LCMS retention time in minutes using indicated Method (A-D);
^{b}Heated to 120° C.

### Example 49—Testing the Biological Activity of Compounds of the Invention

The activity of compounds of the invention was assessed by testing against certain oomycete plant pathogens and representative data are presented in Table 1.

Amended Agar Assay

Testing was carried out on potato dextrose agar (PDA) amended with each compound at a test concentration of 2 ppm. Amended agar was poured into three replicate 9 cm petri dishes. Each replicate dish was inoculated in the centre with a 5 mm agar plug taken from the leading edge of a culture aged between 2 and 7 days old; the age of the culture was dependent on the growth rate of the pathogen being tested. The test pathogen was Pythium ultimum. Plates were incubated at 18° C. and the diameter of each colony measured before growth on the fastest growing plate reached the plate edge. This varied between 2 and 7 days depending on the growth rate of test pathogens. The % reduction in colony growth compared to the control plate was calculated. The results are provided in Table 1, in which D represents no control detected at this concentration; C represents up to 50% control; B from 50 to 99% control; and A represents a control of greater than 99%, i.e. no detectable colony growth.

96 Well Plate Test

Compounds were screened in 96 well plates with 10 compounds per plate. Each compound was screened using agar amended to 2, 0.2, 0.02 and 0.002 ppm, with proline at 50 and 10 ppm and 0.2% DMSO used as controls. Each test concentration and standard was replicated twice on a plate. Compounds were screened against Phytophthora cactorum. The agar used in the test is a 1% potato dextrose agar. 1000 spores/mL agar were added to the appropriate agar.

A ×10 stock solution in 2% DMSO was produced for each dose i.e. 20, 2, 0.2 and 0.02 ppm, and 10 μl of this added to the appropriate wells on the plate. An equivalent amount of 2% DMSO and proline stock at 500 and 100 ppm were added for the controls. To each well 90 μl of the appropriate agar spore suspension was added to give the required final well concentrations. Plates were incubated at room temperature (18° C.) and assessed after 2 to 3 days. The amount of pathogen growth in each well was compared to the DMSO controls and an EC₅₀ concentration was calculated. The results are provided in Table 1 in which G represents no control detected at 2 ppm, F represents an EC₅₀ of ≥2 ppm, and E represents an EC₅₀ of less than 2 ppm.

TABLE 1
% control or EC₅₀

| Com- | Pythium | Phytophthora | Com- | Pythium | Phytophthora |
|---|---|---|---|---|---|
| pound | ultimum | cactorum | pound | ultimum | cactorum |
| 1 | A | E | 2 | A | E |
| 3 | A | E | 4 | A | E |
| 5 | A | E | 6 | B | F |
| 7 | A | E | 8 | A | E |
| 9 | A | E | 10 | A | E |
| 11 | A | E | 12 | A | E |
| 13 | A | E | 14 | A | F |
| 15 | A | E | 16 | A | E |
| 17 | A | E | 18 | A | F |
| 19 | A | E | 20 | A | E |
| 21 | A | E | 22 | B | F |
| 23 | A | F | 24 | A | E |
| 25 | A | E | 26 | A | E |
| 27 | A | E | 28 | B | F |
| 29 | A | E | 30 | A | E |
| 31 | A | E | 32 | A | E |
| 33 | A | E | 34 | A | E |
| 35 | A | E | 36 | A | E |
| 37 | A | E | 38 | A | E |
| 39 | A | E | 40 | A | E |
| 41 | A | E | 42 | A | E |
| 43 | D | G | 44 | C | F |
| 45 | A | E | 46 | A | E |
| 47 | A | E | | | |

Thus, many of the compounds of the invention showed good to excellent control over both of the pathogens tested at 2 ppm (e.g. Examples 1, 2, 3, 4, 5, 7, 8, 9, 10, 11, 12, 13, 15, 16, 17, 19, 20, 21, 24, 25, 26, 27, 29, 30-42, 45-47).

1. A compound of formula (I): 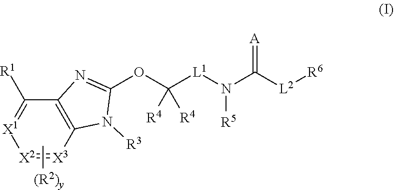
wherein X¹, X² and X³ are each selected from carbon and nitrogen; wherein no more than two of X¹, X² and X³ are nitrogen;
-L¹- is a heteroaryl group independently selected from 6-membered heteroaryl and 5-membered heteroaryl comprising at least one nitrogen atom in the ring, wherein said heteroaryl group is optionally substituted with from 1 to 3 R⁷ groups;
R¹ is independently selected from chloro, bromo, nitro, cyano, NR⁸R⁹, NR⁸CONR⁸R⁸, OR¹⁰, SR⁸, S(O)R⁸, OS(O)₂R⁸, S(O)₂R⁸, S(O)₂NR⁸R⁸, CO₂R⁸, C(O)R⁸, CONR⁸R⁸, C₁-C₆-alkyl, C₂-C₆-alkenyl, C₂-C₆-alkynyl, C₁-C₆-haloalkyl, phenyl, C₃-C₆-cycloalkyl, 3- to 6-membered heterocycloalkyl, 5- or 6-membered heteroaryl, and —O—C₁-C₄-haloalkyl;
R², R⁷ and R¹¹ are each independently at each occurrence selected from: halo, nitro, cyano, NR⁸R⁹, NR⁸CONR⁸R⁸, OR¹⁰, SR⁸, S(O)R⁸, OS(O)₂R⁸, S(O)₂R⁸, S(O)₂NR⁸R⁸, CO₂R⁸, C(O)R⁸, CONR⁸R⁸, C₁-C₆-alkyl, C₂-C₆-alkenyl, C₂-C₆-alkynyl, C₁-C₆-haloalkyl, phenyl, C₃-C₆-cycloalkyl, 3- to 6-membered heterocycloalkyl, 5- or 6-membered heteroaryl, and —O—C₁-C₄-haloalkyl;
R³ is independently selected from 5- or 6-membered heteroaryl, optionally further substituted with from 1 to 4 R¹¹ groups;
R⁴ and R¹³ are each independently at each occurrence selected from H, F, C₁-C₄-alkyl and C₁-C₄-haloalkyl;
or two R⁴ groups together with the carbon to which they are attached form a C₃-C₅-cycloalkyl group;
or two R¹³ groups together with the carbon to which they are attached form a C₃-C₅-cycloalkyl group;
R⁵, R⁸, R¹⁵ and R¹⁷ are each independently at each occurrence selected from H, C₁-C₆-alkyl and C₃-C₆-cycloalkyl;
or two R⁸ groups that are attached to the same nitrogen atom may form a 4- to 7-membered heterocycloalkyl ring;
R⁶ is independently selected from C₁-C₈-alkyl, C₁-C₈-haloalkyl, C₂-C₈-alkenyl, C₂-C₈-alkynyl, C₀-C₃-alkylene-R¹²; and —CR¹³R¹³L³R¹⁴;
R⁹ is independently at each occurrence selected from H, C₃-C₆-cycloalkyl, C₁-C₆-alkyl, C(O)—C₁-C₆-alkyl, C(O)O—C₁-C₆-alkyl and S(O)₂—C₁-C₆-alkyl;
or R⁸ and R⁹ together with the nitrogen to which they are attached form a 4- to 7-membered heterocycloalkyl ring;
R¹⁰ is independently at each occurrence selected from H, C₁-C₆-alkyl, C₃-C₆-alkenyl, C₃-C₆-alkynyl, C₁-C₆-haloalkyl and C₃-C₆-cycloalkyl and C₀-C₃-alkylene-R^{10a}, wherein R^{10a} is independently selected from C₃-C₆-cycloalkyl and 3- to 6-membered heterocycloalkyl;
=A is independently selected from ═O and ═S;
-L²- is absent or is independently selected from —O—, —S—, and —NR¹⁵—;
R¹² and R¹⁶ are each independently at each occurrence selected from C₃-C₆-cycloalkyl, 3- to 6-membered heterocycloalkyl, 5- or 6-membered heteroaryl and phenyl;
R¹³ is independently at each occurrence selected from F, H and C₁-C₄-alkyl;
R¹⁴ is independently selected from H, C₁-C₈-alkyl, C₁-C₈-haloalkyl, C₃-C₈-alkenyl, C₃-C₈-alkynyl and C₀-C₃-alkylene-R¹⁶;
-L³- is independently selected from —O—, —S— and —NR¹⁷—;
y is an integer selected from 0, 1, 2 and 3;
wherein where any R¹-R¹⁷ group is or forms part of an alkyl, alkenyl, alkynyl, haloalkyl, cycloalkyl, heterocycloalkyl, phenyl or heteroaryl group, that alkyl, haloalkyl, cycloalkyl, heterocycloalkyl, phenyl or heteroaryl group is optionally substituted, where chemically possible, by 1 to 5 substituents which are each independently selected at each occurrence from: oxo, ═NR^{a}, ═NOR^{a}, halo, nitro, cyano, NR^{a}R^{b}, NR^{a}S(O)₂R^{a}, NR^{a}C(O)R^{a}, NR^{a}CONR^{a}R^{a}, NR^{a}CO₂R^{a}, OR^{a}, SR^{a}, S(O)R^{a}, S(O)₂R^{a}, S(O)₂NR^{a}R^{a}, CO₂R^{a} C(O)R^{a}, CONR^{a}R^{a}, C₁-C₄-alkyl, C₂-C₄-alkenyl, C₂-C₄-alkynyl and C₁-C₄-haloalkyl; wherein R^{a} is independently at each occurrence selected from H and C₁-C₄ alkyl; and R^{b} is independently at each occurrence selected from H, C₃-C₆-cycloalkyl, C₁-C₄-alkyl, C(O)—C₁-C₄-alkyl and S(O)₂—C₁-C₄-alkyl;
or an agronomically acceptable salt or N-oxide thereof.

2. A compound of claim 1, wherein compound of formula (I) is a compound of formula (VI): 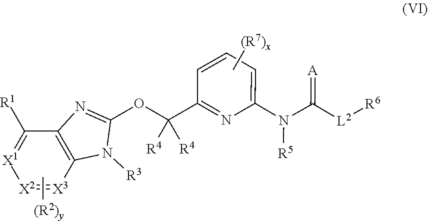
wherein x is an integer independently selected from 0, 1, 2 and 3.

3. A compound of claim 1, wherein the compound of formula (I) is a compound of formula (X): 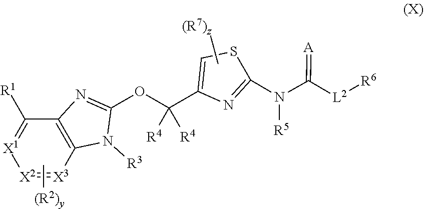
wherein z is an integer independently selected from 0 and 1.

4. A compound of claim 1, wherein each of X¹, X² and X³ are carbon.

5. A compound of claim 1, wherein R⁴ is at each occurrence H.

6. A compound of claim 1, wherein R³ is substituted at a position adjacent to the point of connection of R³ to the rest of the molecule with an R^{11b} group, wherein R^{11b} is selected from C₁-C₄-alkyl, C₂-C₄-alkenyl, C₂-C₄-alkynyl, phenyl, C₃-C₆-cycloalkyl, 3- to 6-membered heterocycloalkyl, 5- or 6-membered heteroaryl and C₁-C₄-haloalkyl.

7. A compound of claim 6, wherein R³ has the structure: 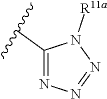
wherein R^{11a} is selected from C₁-C₄-alkyl, C₃-C₄-alkenyl, C₃-C₄-alkynyl, phenyl, C₃-C₆-cycloalkyl, 3- to 6-membered heterocycloalkyl, 5- or 6-membered heteroaryl and C₁-C₄-haloalkyl.

8. A compound of claim 7, wherein R^{11a} is C₁-C₄-alkyl.

9. A compound of any one claim 1, wherein y is 0.

10. A compound of claim 1, wherein =A is ═O.

11. A compound of claim 1, wherein R⁵ is H.

12. A compound of claim 1, wherein R¹ is selected from Cl, OR¹⁰ and C₁-C₄-alkyl.

13. A compound of claim 1, wherein the compound of formula (I) is selected from:

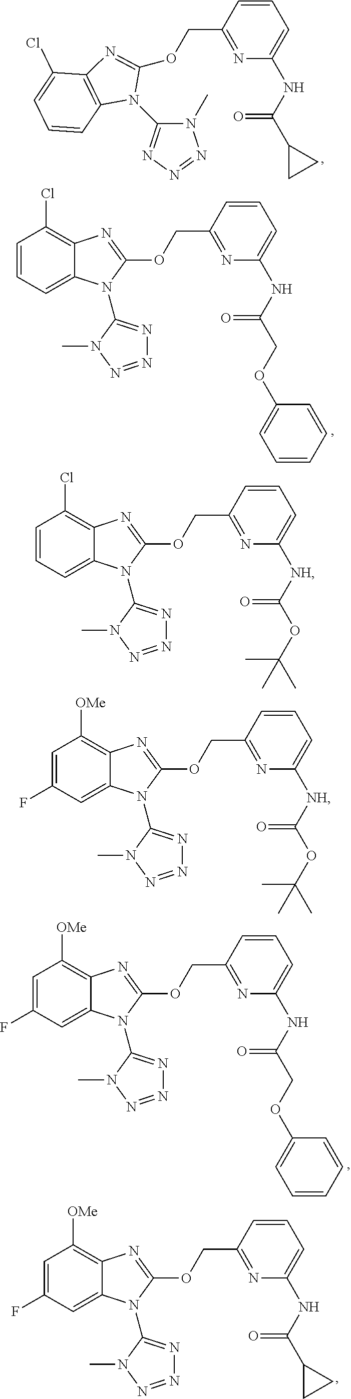

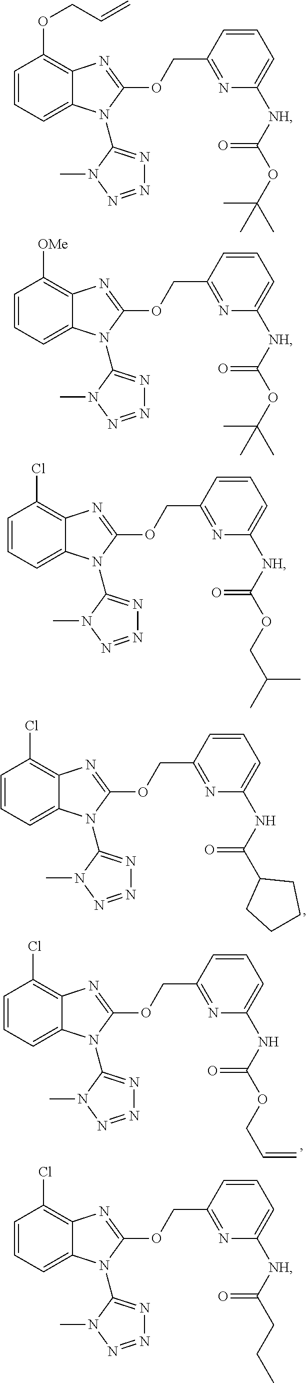

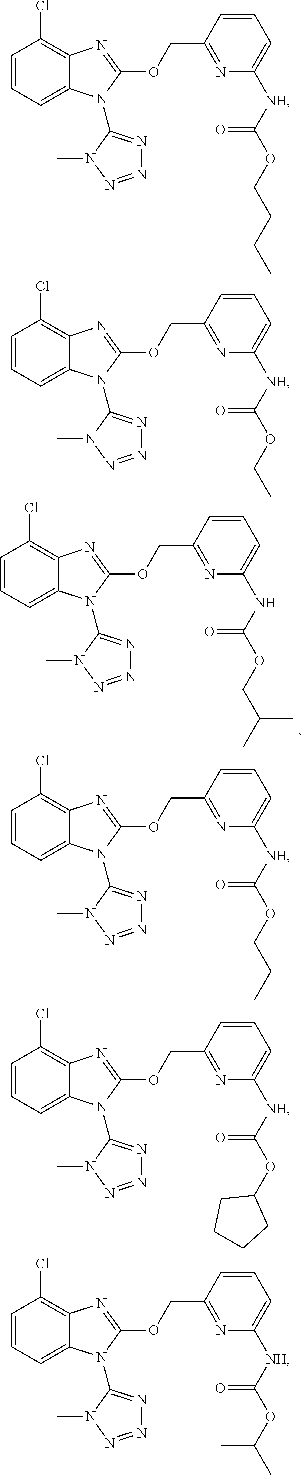

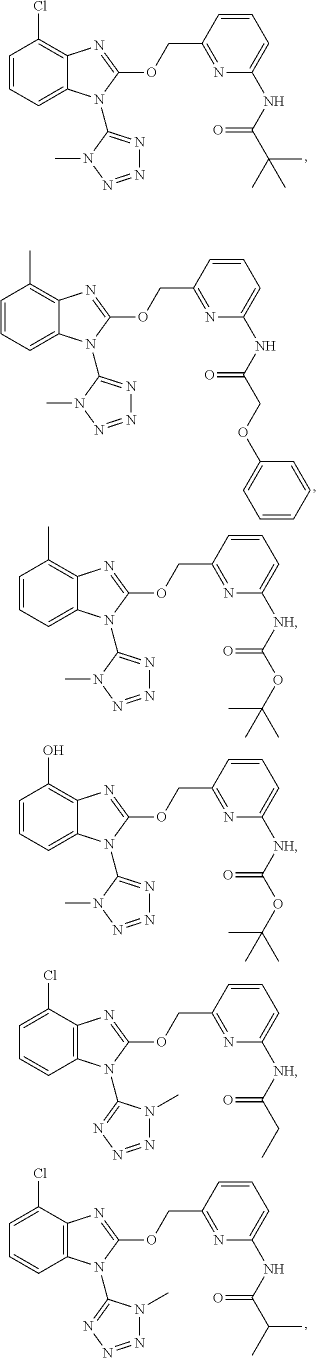

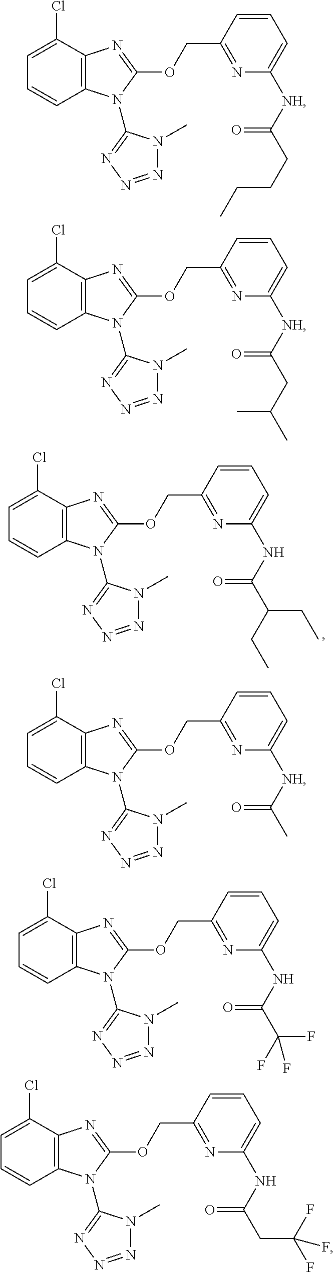

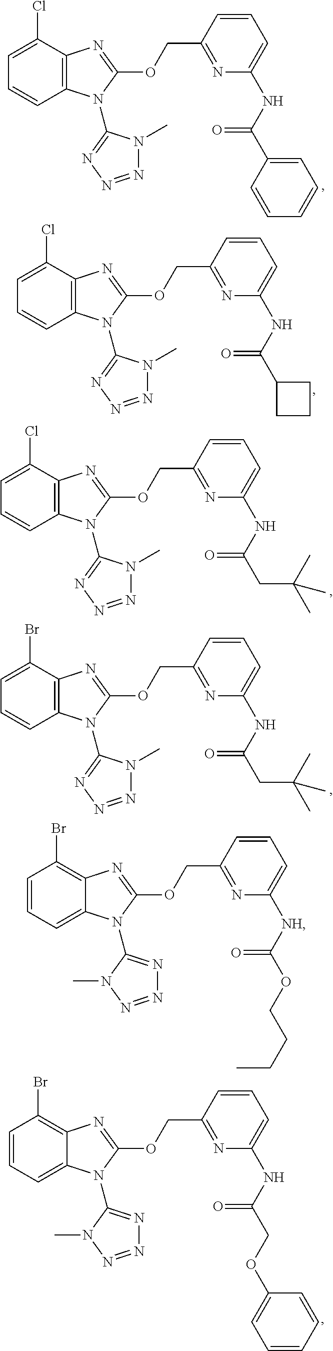

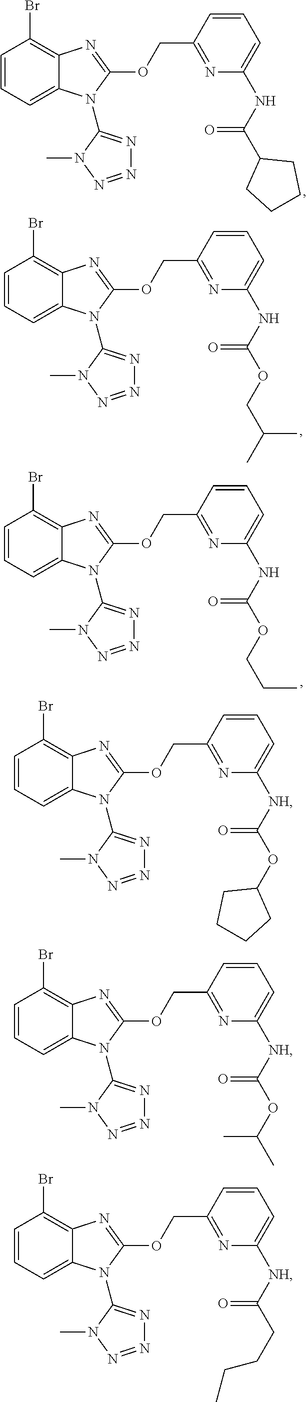

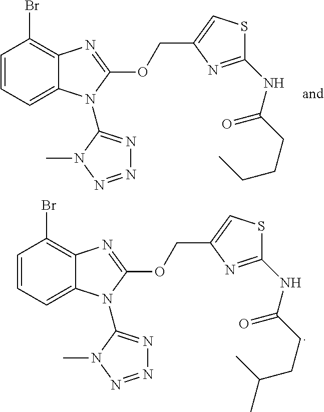

14. A method for controlling fungal diseases, the method comprising applying an agronomically effective and substantially non-phytotoxic quantity of a compound of claim 1 to seeds of plants, to plants themselves or to an area where it is intended that plants will grow.

15. A use of a compound of claim 1 to control fungal diseases.

16. A fungicidal composition comprising an effective and non-phytotoxic amount of an active compound of claim 1.
